(19) United States
(12) Patent Application Publication
(10) Pub. No.: US 2025/0170114 A1
(43) Pub. Date: May 29, 2025

(54) METHODS OF TREATMENT USING T-TYPE CALCIUM CHANNEL MODULATORS

(71) Applicant: Praxis Precision Medicines, Inc., Boston, MA (US)

(72) Inventor: Bernard Ravina, Newton, MA (US)

(21) Appl. No.: 18/834,466

(22) PCT Filed: Feb. 3, 2023
(86) PCT No.: PCT/US2023/061973

§ 371 Date: Jul. 30, 2024

Related U.S. Application Data
(60) Provisional application No. 63/306,340, filed on Feb. 3, 2022.

(51) Int. Cl. A61K 31/445; A61K 9/00; A61K 31/138; A61K 31/36; A61K 31/513

(52) U.S. Cl. CPC A61K 31/445; A61K 9/0053; A61K 31/138; A61K 31/36; A61K 31/513

(57) ABSTRACT

Described herein, in part, are methods useful for treating a disease or condition relating to aberrant function or activity of a T-type calcium channel, such as essential tremor, in a subject in need thereof, comprising administering a T-type calcium channel inhibitor in combination with at least one of propranolol, primidone, topiramate, or a pharmaceutically acceptable salt thereof. Also disclosed herein are compositions comprising the T-type calcium channel inhibitor and at least one of propranolol, primidone, topiramate, or a pharmaceutically acceptable salt thereof.

### FIELD OF THE DISCLOSURE

Disclosed herein are methods useful for treating a disease or condition related to aberrant function of T-type calcium channels, such as essential tremor, in a subject in need thereof, comprising administering a T-type calcium channel inhibitor in combination with at least one of propranolol, primidone, topiramate, or a pharmaceutically acceptable salt thereof.

### BACKGROUND

Essential tremor (ET) is the most common movement disorder, affecting up to 7 million patients in the United States alone. As an action tremor, ET manifests itself during voluntary movements, thereby interfering with basic life functions and causing direct disability and impaired job performance. Approximately 50% of ET patients seek pharmacological therapy; however, tremor is only partially managed in these patients, with almost half discontinuing medications due to limited efficacy and/or poor tolerability.

Essential tremor, which is also known as benign essential tremor, is the most common type of tremor. Essential tremor may be mild and nonprogressive in some patients, and in some patients, it may be slowly progressive, starting on one side of the body but typically affecting both sides. The hands are most often affected, but the head, voice, tongue, legs, and trunk may also be involved. Tremor frequency may decrease as the person ages, but severity may also increase. Heightened emotion, stress, fever, physical exhaustion, or low blood sugar may trigger tremors and/or increase their severity. Symptoms generally evolve over time and can be both visible and persistent following onset.

Currently, the beta blocker propranolol, approved by the FDA in 1967, remains the only approved therapy for ET in the United States. A non-selective beta blocker, propranolol is contraindicated for individuals with certain respiratory or cardiac issues, which are common comorbidities in the age group frequently affected by ET. Primidone, an anticonvulsant, may be used to control ET, but it requires slow titration over six to eight weeks and can cause sedation and balance issues while accelerating osteoporosis with long-term use. Topiramate is another example of an anticonvulsant that may be used to treat ET, but patients often report undesirable side effects, including visual problems, cognitive problems, loss of appetite, and tingling sensations.

As a last-line therapy, several thousand ET patients in the United States opt for invasive surgery each year. Interventions include Gamma Knife® surgery and focused ultrasound thalamotomy, where part of the thalamus involved in the cerebello-thalamo-cortical (CTC) pathway is ablated, and deep brain stimulation (DBS), where an electrode is implanted into the brain. These procedures are generally effective but may be associated with significant side effects and risk. Many patients who are eligible for surgical therapies do not elect to have these procedures.

Therefore, a significant unmet need remains for the millions of ET patients who are not currently receiving treatment for their ET or who are underserved by existing treatment options. One recent strategy for treating ET is through the administration of T-type calcium channel inhibitors. There is a large body of clinical, preclinical, and genetic evidence that points to the involvement of T-type calcium channels in the CTC pathway as a major driver of ET. Successful development of T-type calcium channel modulators in ET likely requires a pharmacokinetic (PK) profile with a blunted Cmax and thoughtful clinical trial design and endpoint selection. To this end, the compound of Formula (I), depicted below, is being developed for the treatment of ET.

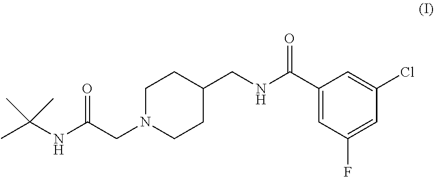 [SMILES: CC(C)(C)NC(=O)CN1CCC(CNC(=O)c2cc(F)cc(Cl)c2)CC1]

A compound of Formula (I) is described, for example, in PCT Publication WO2009/146540, incorporated by reference herein. The safety profile, efficacy, tolerability, and pharmacokinetics of the compound of Formula (I) in modified release formulations with and without titration has been assessed, as described in PCT Publication WO2021/222342, incorporated by reference herein. Given the potential of the compound of Formula (I) to treat ET, strategies for further developing the compound of Formula (I) to treat ET is an important goal.

### SUMMARY

Described herein are methods of treating a disease or condition relating to aberrant function or activity of a T-type calcium channel in a subject in need thereof, comprising administering to the subject a compound of Formula (I):

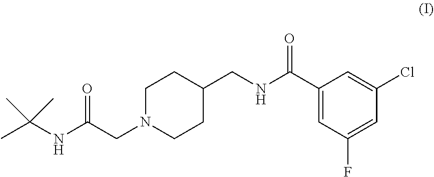 [SMILES: CC(C)(C)NC(=O)CN1CCC(CNC(=O)c2cc(F)cc(Cl)c2)CC1]

or a pharmaceutically acceptable salt thereof in combination with at least one of propranolol, primidone, topiramate, or pharmaceutically acceptable salts thereof. In some variations, the compound of Formula (I) or a pharmaceutically acceptable salt thereof is administered in combination with propranolol or a pharmaceutically acceptable salt thereof.

In some embodiments, the disease or condition relating to aberrant function or activity of a T-type calcium channel is essential tremor (ET). Therefore, also disclosed herein are methods of treating ET in a subject in need thereof, comprising administering to the subject a compound of Formula (I) or a pharmaceutically acceptable salt thereof in combination with at least one of propranolol, primidone, topiramate, or pharmaceutically acceptable salts thereof.

In some embodiments, the compound used for treating the disease or condition relating to aberrant function or activity of a T-type calcium channel, such as ET, is the hydrochloride (HCl) salt of the compound of Formula (I), depicted below:

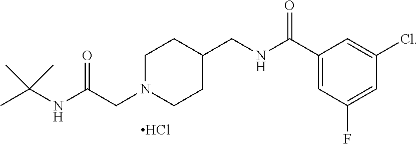 [SMILES: CC(C)(C)NC(=O)CN1CCC(CNC(=O)c2cc(F)cc(Cl)c2)CC1 ; Cl]

In some embodiments, the compound used for treating the disease or condition relating to aberrant function or activity of a T-type calcium channel, such as ET, is a crystalline form of the compound of Formula (I), e.g., crystalline HCl salt of the compound of Formula (I). In some embodiments, the compound used for treating the disease or condition relating to aberrant function or activity of a T-type calcium channel, such as ET, is a polymorph of a crystalline salt of the compound of Formula (I), e.g., a polymorph of a crystalline HCl salt of the compound of Formula (I), e.g., as described in WO 2021007487A1, the entire contents of which are hereby incorporated herein by reference.

In some embodiments, propranolol is administered as an HCl salt. In some embodiments, propranolol or a pharmaceutically acceptable salt thereof is administered as an (S)-enantiomer. In particular embodiments, (S)-propranolol hydrochloride is administered in combination with the compound of Formula (I) or a pharmaceutically acceptable salt thereof (e.g., the HCl salt). In other embodiments, (R)-propranolol hydrochloride is administered in combination with the compound of Formula (I) or a pharmaceutically acceptable salt thereof (e.g., the HCl salt). In some embodiments, compound of Formula (I) or a pharmaceutically acceptable salt thereof, e.g., the HCl salt of compound of Formula (I) may be referred to herein in the examples as the “study drug”.

In some embodiments, the subject suffering from the disease or condition relating to aberrant function or activity of a T-type calcium channel, such as ET, had previously been on a dosing regimen of propranolol or a pharmaceutically acceptable salt thereof prior to administration of the compound of Formula (I) or a pharmaceutically acceptable salt thereof. In some embodiments, the subject suffering from the disease or condition relating to aberrant function or activity of a T-type calcium channel, such as ET, had previously been on a dosing regimen of primidone or a pharmaceutically acceptable salt thereof prior to administration of the compound of Formula (I) or a pharmaceutically acceptable salt thereof, and in some embodiments, the subject suffering from the disease or condition relating to aberrant function or activity of a T-type calcium channel, such as ET, had previously been on a dosing regimen of topiramate or a pharmaceutically acceptable salt thereof prior to administration of the compound of Formula (I) or a pharmaceutically acceptable salt thereof.

In some embodiments, a subject who had previously been on a dosing regimen of propranolol, primidone, topiramate, or a pharmaceutically acceptable salt thereof had an active prescription for propranolol, primidone, topiramate, or a pharmaceutically acceptable salt thereof, and was directed by a physician to take propranolol, primidone, topiramate, or a pharmaceutically acceptable salt thereof. After the start of administration of the compound of Formula (I) to a subject who had previously been on a dosing regimen of propranolol, primidone, topiramate, or a pharmaceutically acceptable salt thereof, the subject's active prescription for propranolol, primidone, topiramate or a pharmaceutically acceptable salt thereof may be terminated, or may be modified to adjust the dosing regimen or a dosage amount of propranolol, primidone, topiramate or a pharmaceutically acceptable salt thereof, or the prescription may continue unchanged. In some embodiments, a subject who had previously been on a dosing regimen of propranolol, primidone, topiramate or a pharmaceutically acceptable salt thereof is administered the compound of Formula (I), while administration of propranolol, primidone, topiramate or a pharmaceutically acceptable salt thereof to the subject is terminated. In some embodiments, a subject who had previously been on a dosing regimen of propranolol, primidone, topiramate or a pharmaceutically acceptable salt thereof is administered the compound of Formula (I) in combination with propranolol, primodone, topiramate or a pharmaceutically acceptable salt thereof.

As disclosed herein, ET patients who were taking propranolol hydrochloride responded favorably (e.g., reduced tremor symptoms) to the HCl salt of the compound of Formula (I) in a phase 2 clinical trial designed to evaluate efficacy, safety, and tolerability of the combination therapy without significant side effects. Therefore, in accordance with the disclosure, the compound of Formula (I) or a pharmaceutically acceptable salt thereof can serve as an adjunctive therapy to at least one of propranolol, primidone, topiramate, or a pharmaceutically acceptable salt thereof. In some variations, the propranolol, primidone, topiramate, or pharmaceutically acceptable salt thereof is dosed chronically or as needed (PRN) in the subject. For example, in certain embodiments, the propranolol, primidone, topiramate, or pharmaceutically acceptable salt thereof may be administered PRN to a subject having mild ET symptoms before a situation/action in anticipation of the tremor(s) that the situation/action will induce. In certain embodiments, the propranolol, primidone, topiramate, or pharmaceutically acceptable salt thereof may be administered PRN to a subject based on the subject's self-determination that the propranolol, primidone, topiramate, or pharmaceutically acceptable salt thereof will treat ET as needed. A subject's self-determination may be based, for example, on self-assessment of symptoms and/or on data provided by assessment devices, e.g., wearable technology.

In one aspect, the present disclosure provides a method of treating a disease or condition relating to aberrant function or activity of a T-type calcium channel, such as ET, in a subject in need thereof, the method comprising administering (e.g., once, twice, or three times) daily to the subject up to about 120 mg (e.g., from about 5 mg to about 120 mg, from about 10 mg to about 120 mg, from about 15 mg to about 120 mg, from about 20 mg to about 120 mg, from about 20 mg to about 100 mg, or from about 20 mg to about 80 mg) of the compound of Formula (I) or a pharmaceutically acceptable salt thereof (e.g., the HCl salt), wherein the subject is concurrently taking at least one of propranolol, primidone, topiramate, or a pharmaceutically acceptable salt thereof (e.g., propranolol HCl), i.e., the subject is being administered the compound of Formula (I) in combination with at least one of propranolol, primidone, topiramate, or a pharmaceutically acceptable salt thereof as described herein. In some embodiments, the subject is taking (S)-propranolol HCl.

In some embodiments where the subject is or was on a regimen of at least one of propranolol, primidone, topiramate, or a pharmaceutically acceptable salt thereof prescribed prior to the compound of Formula (I) or a pharmaceutically acceptable salt thereof, the subject remains on the same dosing regimen of propranolol, primidone, topiramate, or a pharmaceutically acceptable salt thereof that the subject was on prior to the administration of the compound of Formula (I) or a pharmaceutically acceptable salt thereof (e.g. the HCl salt). In other embodiments, the subject is administered a lower dose of propranolol, primidone, topiramate, or a pharmaceutically acceptable salt thereof than the subject was on prior to the administration of the compound of Formula (I) or a pharmaceutically acceptable salt thereof.

In another embodiment, the method of treating the disease or condition relating to aberrant function or activity of a T-type calcium channel, such as ET, in a subject in need thereof comprises administering at least one of propranolol, primidone, topiramate, or a pharmaceutically acceptable salt thereof (e.g., propranolol HCl) and administering a compound of Formula (I) or a pharmaceutically acceptable salt thereof (e.g., the HCl salt) on a schedule that involves titrating the dose of the compound of Formula (I) or a pharmaceutically acceptable salt thereof up to a desired dosing level. For instance, in some embodiments, the dose of the compound of Formula (I) or a pharmaceutically acceptable salt thereof can be titrated from a dose starting at about 5 mg, about 10 mg, or about 20 mg daily up to a dose of about 120 mg daily. In some embodiments, the dose of the compound of Formula (I) or a pharmaceutically acceptable salt thereof can be titrated from a dose starting at about 10 mg daily up to a dose of about 120 mg daily (e.g., about 20 mg daily, about 60 mg daily, about 80 mg daily, or about 100 mg daily). In some embodiments, the dose of the compound of Formula (I) or a pharmaceutically acceptable salt thereof can be titrated from a dose starting at about 20 mg daily up to a dose of about 120 mg daily (e.g., about 40 mg daily, about 60 mg daily, about 80 mg daily, or about 100 mg daily). In some embodiments, each dose is administered in a modified or extended release composition, as set forth herein.

In another aspect, the disclosure provides a single oral composition comprising the compound of Formula (I) or a pharmaceutically acceptable salt thereof (e.g., the HCl salt) and at least one of propranolol, primidone, topiramate, or a pharmaceutically acceptable salt thereof (e.g., propranolol HCl). In certain embodiments, the composition comprising the compound of Formula (I) or a pharmaceutically acceptable salt thereof (e.g., the HCl salt) and at least one of propranolol, primidone, topiramate, or a pharmaceutically acceptable salt thereof may be formulated for once daily administration. In some variations, the propranolol is (S)-propranolol, (R)-propranolol, or a mixture of (S)- and (R)-propranolol. In certain embodiments, the propranolol in the composition is (S)-propranolol HCl. In certain embodiments, the propranolol in the composition is (R)-propranolol HCl. In yet other embodiments, the propranolol in the composition is mixture of (S)- and (R)-propranolol HCl. In certain embodiments, the composition includes at least one of propranolol, primidone, topiramate, or a pharmaceutically acceptable salt thereof and the compound of Formula (I) or a pharmaceutically acceptable salt thereof, wherein the compounds are present in a ratio of from about 1:10 to about 10:1, by weight in the composition. For instance, the ratio between propranolol, primidone, topiramate, or a pharmaceutically acceptable salt thereof and the compound of Formula (I) or a pharmaceutically acceptable salt thereof can be about 1:10, 1:5, 1:4, 1:3, 1:2, 1:1, 2:1, 3:1, 4:1, 5:1 or 10:1, by weight in the composition. It will be understood that all weights are based on the weight of the free base of the compound of Formula (I) and propranolol, primidone, or topiramate, respectively.

Other objects and advantages will become apparent to those skilled in the art from a consideration of the ensuing disclosure.

### BRIEF DESCRIPTION OF THE FIGURES

FIG. 1 is a graph showing the percent change in modified activities of daily living (ADL) scores as compared to baseline combined upper limb (CUL) scores from Part B participants, as discussed in Example 1.

FIG. 2 is a graph showing the percent change in Essential Tremor Rating Assessment Scale (TETRAS) CUL scores (top) and TETRAS ADL scores (bottom) as compared to baseline CUL scores from Part B participants, as discussed in Example 1.

FIG. 3 is a graph showing the percent change in tremor amplitude as compared to baseline CUL scores from Part A participants (left) and Part B participants (right), as discussed in Example 1.

FIG. 4, Panel A is a bar graph showing tremor power measured in the 8-13 Hz band in harmaline-treated rats that were administered 1 mg/kg of the compound of Formula (I) alone, or in combination with 1 mg/kg or 3 mg/kg propranolol. FIG. 4, Panel B is a bar graph showing tremor power measured in the 6-15 Hz band in harmaline-treated rats that were administered compound of 1 mg/kg of the compound of Formula (I) (the “study drug”) alone, or in combination with 1 mg/kg or 3 mg/kg propranolol.

FIG. 5 is a bar graph showing sLMA as total distance travelled (in mm) measured in rats treated with the compound of Formula (I) (the “study drug”) and propranolol alone or in combination.

FIG. 6 is a graph showing sLMA as total distance travelled (in mm) over time measured in rats treated with the compound of Formula (I) (the “study drug”) and propranolol alone or in combination.

FIG. 7 is a bar graph showing the tremor score measured in rats that were administered 30 mg/kg harmaline and also administered 10 mg/kg propranolol or 1 mg/kg, 3 mg/kg or 10 mg/kg study drug, i.e., compound of Formula (I).

FIG. 8 is a bar graph showing the traveling distance in 0-5 minutes (mm) measured in rats that were administered 30 mg/kg harmaline and also administered 10 mg/kg propranolol or 1 mg/kg, 3 mg/kg or 10 mg/kg study drug, i.e., compound of Formula (I).

FIG. 9 is a bar graph showing the mean of fall latency measured in rats that were administered 30 mg/kg harmaline and also administered 10 mg/kg propranolol or 1 mg/kg, 3 mg/kg or 10 mg/kg study drug, i.e., compound of Formula (I).

FIG. 10 is a bar graph showing mean latency to fall (sec) measured in rats that were administered 30 mg/kg harmaline and also administered 10 mg/kg propranolol or 1 mg/kg, 3 mg/kg or 10 mg/kg study drug, i.e., compound of Formula (I).

FIG. 11 is a bar graph showing traveling distance in 0-30 minutes (mm) of rats treated with harmaline (30 mg/kg) and also treated with 3 mg/kg propranolol and 1 mg/kg and 3 mg/kg of the study drug, i.e., compound of Formula (I).

FIG. 12 is a bar graph showing traveling distance in 0-30 minutes (mm) of rats treated with harmaline (30 mg/kg) and also treated with 10 mg/kg propranolol and 1 mg/kg and 3 mg/kg of the study drug, i.e., compound of Formula (I).

FIG. 13 is a bar graph showing the same data as in FIGS. 11 and 12, and also showing the results of statistical comparison between different groups of rats.

### DETAILED DESCRIPTION

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

The term “about” as used herein refers to the usual error range for the respective value readily known in this technical field. Reference to “about” a value or parameter herein includes (and describes) variations that are directed to that value or parameter per se. For example, description referring to “about X” includes description of “X.”

The terms “disease”, “disorder”, and “condition” are used interchangeably herein.

The term “modified-release polymer” refers to a polymer that is used in a formulation (e.g., tablets and capsules) to modify the release rate of the drug upon the administration to a subject. For example, a modified-release polymer is used to dissolve a drug over time in order to be released more slowly and/or more steadily into the bloodstream. For example, a modified-release polymer is a controlled-release polymer. For example, a modified-release polymer or a controlled-release polymer is a hydroxy-propyl methylcellulose (HPMC) polymer. In some embodiments, a modified-release polymer may include hydrophilic matrix polymers (e.g., hypromellose, HPMC), hydrophobic matrix polymers (e.g., ethyl cellulose, ethocel), or polyacrylate polymers (e.g., Eudragit RL100, Eudragit RS100).

The term “pharmaceutically acceptable salt” refers to those salts that are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, stereoisomers thereof (e.g., enantiomers, diastereomers) and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “refractory” refers to a disease, disorder, or condition that does not readily yield or respond to therapy or treatment, or is not controlled by a therapy or treatment. In some embodiments, a disease, disorder, or condition described herein is refractory (e.g., refractory epilepsy or refractory absence seizures) and does not respond to standard therapy or treatment. For instance, in accordance with the disclosure, in some embodiments, the patients who are taking propranolol no longer respond to propranolol.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human” and “patient” are used interchangeably herein, while the term “subject” may refer to human or non-human animals.

As used herein, a “subject in need thereof” is a subject who has a disease, disorder or condition relating to aberrant function or activity of a T-type calcium channel. In some embodiments, a “subject in need thereof” is a subject who has essential tremor (ET). In some embodiments, a “subject in need thereof” is a subject who has ET that is or has become refractory to treatment with propranolol, primidone, topiramate or a pharmaceutically acceptable salt thereof.

The term “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of a disease or condition, or enhances the therapeutic efficacy of another therapeutic agent.

The terms “treat,” “treating,” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment” or “prevention” of the specified disease, disorder or condition). The terms “treat”, “treating,” and “treatment” also refer to reversing, alleviating, arresting or ameliorating a disease, e.g., ET, or at least one of the clinical symptoms of a disease, e.g., ET, or inhibiting the progress of a disease or at least one of the clinical symptoms of the disease, e.g., ET.

The term “coating” as used herein refers to an excipient to protect tablet ingredients from deterioration by moisture in the air and/or make large or unpleasant-tasting tablets easier to swallow.

The term “diluent” as used herein refers to an excipient used to increase weight and improve content uniformity. For example, diluents include cellulose derivatives (e.g., microcrystalline cellulose), starches (e.g., hydrolyzed starches and partially pregelatinized starches), anhydrous lactose, lactose monohydrate, di-calcium phosphate (DCP), and sugar alcohols (e.g., sorbitol, xylitol and mannitol).

The term “glidant” as used herein refers to an excipient used to promote powder flow by reducing interparticle friction and cohesion. For example, glidants include fumed silica (e.g., colloidal silicon dioxide), talc, and magnesium carbonate.

The term “lubricant” as used herein refers to an excipient used to prevent ingredients from clumping together and/or from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

In some embodiments, methods provided by the present disclosure comprise administering to a subject in need thereof a compound of Formula (I):

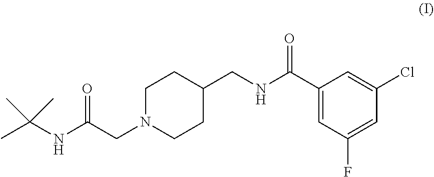 [SMILES: CC(C)(C)NC(=O)CN1CCC(CNC(=O)c2cc(F)cc(Cl)c2)CC1]

or a pharmaceutically acceptable salt thereof in combination with at least one of propranolol, primidone, topiramate, or pharmaceutically acceptable salts thereof. As used herein, the term “in combination” refers to administration to a subject in need thereof of a compound of Formula (I) or a pharmaceutically acceptable salt thereof and of at least one of propranolol, primidone, topiramate, or pharmaceutically acceptable salts thereof whereby the subject has an active prescription for the compound of Formula (I) or a pharmaceutically acceptable salt thereof and an active prescription for at least one of propranolol, primidone, topiramate, or a pharmaceutically acceptable salt thereof, and is being directed by a physician to take the compound of Formula (I) of a pharmaceutically acceptable salt thereof and at least one of propranolol, primidone, topiramate, or a pharmaceutically acceptable salt thereof. In some embodiments, the term “in combination” also refers to administration to a subject in need thereof of a compound of Formula (I) or a pharmaceutically acceptable salt thereof and of at least one of propranolol, primidone, topiramate, or pharmaceutically acceptable salts thereof over the same period of time. In some embodiments, the compound of Formula (I) of a pharmaceutically acceptable salt thereof and at least one of propranolol, primidone, topiramate, or a pharmaceutically acceptable salt thereof may be administered in combination to a subject in need thereof each according to the same administration schedule or each according to different administration schedules. For example, in some embodiments, the compound of Formula (I) of a pharmaceutically acceptable salt thereof and at least one of propranolol, primidone, topiramate, or a pharmaceutically acceptable salt thereof may be each be administered to a subject in need thereof over the same period of time once daily, e.g., in the morning. In other embodiments, the compound of Formula (I) of a pharmaceutically acceptable salt thereof may be administered to a subject once daily, e.g., in the morning, and at least one of propranolol, primidone, topiramate, or a pharmaceutically acceptable salt thereof may be administered to a subject in need thereof two or three times daily over the same period of time. In some embodiments, the compound of Formula (I) of a pharmaceutically acceptable salt thereof and at least one of propranolol, primidone, topiramate, or a pharmaceutically acceptable salt thereof may be administered to a subject in need thereof simultaneously as a part of a new pharmaceutical composition. In other embodiments, the compound of Formula (I) of a pharmaceutically acceptable salt thereof and at least one of propranolol, primidone, topiramate, or a pharmaceutically acceptable salt thereof may be administered in combination simultaneously, or within several minutes or several hours to a subject in need thereof as parts of different pharmaceutical compositions.

These and other exemplary substituents are described in more detail in the Detailed Description, Examples, and Claims. The invention is not intended to be limited in any manner by the above exemplary listing of substituents.

### II. Methods of Treating T-Type Calcium Channel Diseases or Conditions

In one aspect, provided are methods of treating a disease or condition relating to aberrant function or activity of a T-type calcium channel in a subject in need thereof, comprising administering a compound of Formula (I):

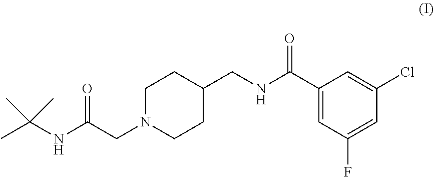 [SMILES: CC(C)(C)NC(=O)CN1CCC(CNC(=O)c2cc(F)cc(Cl)c2)CC1]

or a pharmaceutically acceptable salt thereof in combination with at least one of propranolol, primidone, topiramate, or pharmaceutically acceptable salts thereof.

In certain embodiments, disclosed herein are methods of treating ET in a subject in need thereof comprising administering to the subject a compound of Formula (I) or a pharmaceutically acceptable salt thereof in combination with at least one of propranolol, primidone, topiramate, or pharmaceutically acceptable salts thereof.

The disclosure also includes administering deuterium-enriched compounds of Formula (I) or pharmaceutically acceptable salts thereof in combination with at least one of propranolol, primidone, topiramate, or pharmaceutically acceptable salts thereof (e.g., propranolol HCl) to treat a disease or condition relating to aberrant function or activity of a T-type calcium channel, such as ET, in a subject in need thereof. For instance, in one embodiment, the deuterium-enriched compound has a Formula (II):

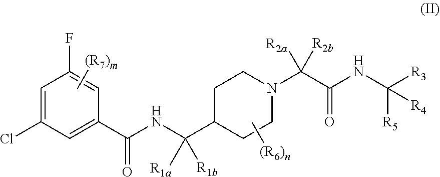

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Ra)₃, wherein each Ra is independently hydrogen or deuterium;
n is an integer selected from 0 to 9;
m is an integer selected from 0 to 3.

In another embodiment, the deuterium-enriched compound has a Formula (II-A),

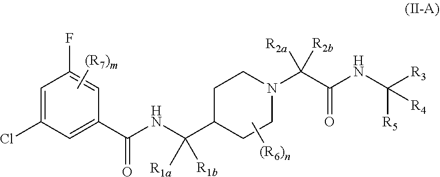

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Ra)₃, wherein each Ra is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Ra is deuterium.

In another embodiment, the deuterium-enriched compound has a Formula (II-B),

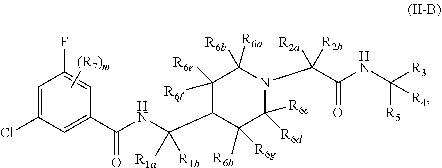

or a pharmaceutically acceptable salt thereof, wherein:
each of R₁ₐ, R_{1b}, R₂ₐ, R_{2b}, and R₇ is independently hydrogen or deuterium;
each of each of R₃, R₄, and R₅ is —C(Ra)₃, wherein each Ra is independently hydrogen or deuterium;
m is an integer selected from 0 to 3;
at least one of R₆ₐ, R_{6b}, R_{6c}, and R₆ₐ is deuterium; and
each of R₆ₑ, R_{6f}, R_{6g}, R₆ₕ is independently hydrogen or deuterium.

In another embodiment, the deuterium-enriched compound has a formula selected from the group consisting of:

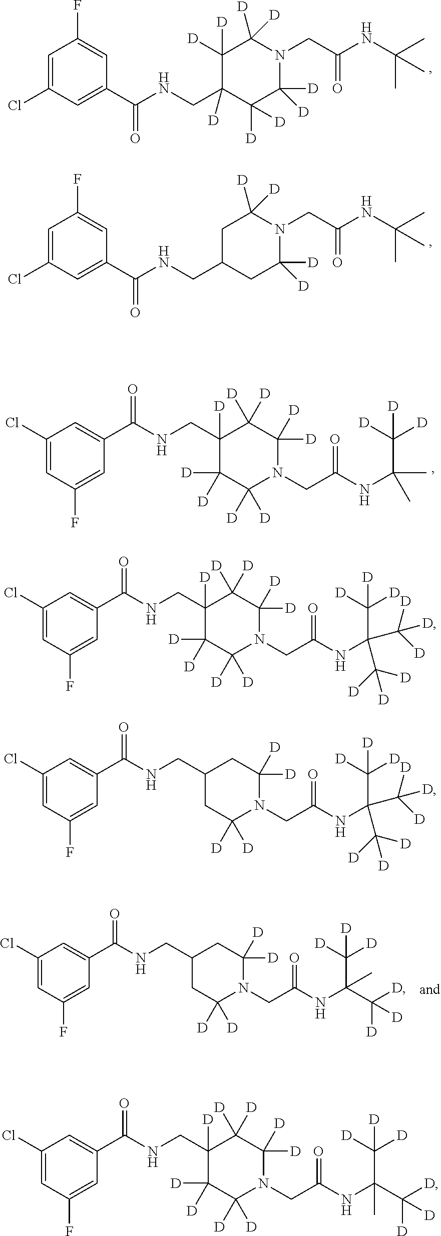

or a pharmaceutically acceptable salt thereof.

It will be understood that any reference to the compound of Formula (I) for the treatment of essential tremor applies equally to the deuterium-enriched compounds described herein (e.g., compounds of Formula (II), Formula (II-A), and Formula (II-B) and the compounds in the preceding paragraph).

In some embodiments, the compound used for treating the disease or condition relating to aberrant function or activity of a T-type calcium channel, such as ET, is the HCl salt of the compound of Formula (I), depicted below:

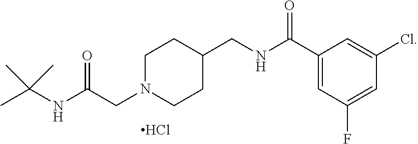 [SMILES: CC(C)(C)NC(=O)CN1CCC(CNC(=O)c2cc(F)cc(Cl)c2)CC1 ; Cl]

In one aspect, the present disclosure provides a method of treating the disease or condition relating to aberrant function or activity of a T-type calcium channel, such as ET, in a subject in need thereof, the method comprising administering (e.g., once, twice, or three times) daily to the subject up to about 120 mg (e.g., from about 5 mg to about 120 mg, from about 10 mg to about 120 mg, from about 15 mg to about 120 mg, from about 20 mg to about 120 mg, from about 20 mg to about 100 mg, or from about 20 mg to about 80 mg) of the compound of Formula (I) or a pharmaceutically acceptable salt thereof (e.g., the HCl salt), wherein the subject is concurrently taking at least one of propranolol, primidone, topiramate, or a pharmaceutically acceptable salt thereof (e.g., propranolol HCl), i.e., the subject is being administered the compound of Formula (I) in combination with at least one of propranolol, primidone, topiramate, or a pharmaceutically acceptable salt thereof as described herein.

In some such embodiments, the subject had been taking a dose of from about 10 mg to about 1000 mg/daily of at least one of propranolol, primidone, topiramate, or a pharmaceutically acceptable salt thereof, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. For example, in certain embodiments, the subject had been taking a dose of from about 20 mg to about 200 mg/daily of propranolol or a pharmaceutically acceptable salt thereof, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 20 mg to about 100 mg/daily of propranolol or a pharmaceutically acceptable salt thereof, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 20 mg to about 40 mg/daily of propranolol or a pharmaceutically acceptable salt thereof, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In certain embodiments, the subject had been taking a dose of from about 10 mg to about 1000 mg/daily of primidone, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 12.5 mg to about 750 mg/daily of primidone, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 50 mg to about 250 mg/daily primidone, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 10 mg to about 400 mg/daily of topiramate, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 25 mg to about 200 mg/daily of topiramate, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof. In other such embodiments, the subject had been taking a dose of from about 10 mg to about 50 mg/daily of topiramate, prior, e.g., at least 1 year, at least 6 months, at least 3 months, at least 2 months, at least 1 month, at least 2 weeks or at least 1 week prior to the administration of the compound of Formula (I) or a pharmaceutically acceptable salt thereof.

In some embodiments where the subject was on a regimen of at least one of propranolol, primidone, topiramate, or a pharmaceutically acceptable salt thereof prior to being administered the compound of Formula (I) or a pharmaceutically acceptable salt thereof, the subject can remain on the same dosing regimen of the propranolol, primidone, topiramate, or a pharmaceutically acceptable salt thereof that the subject was on prior to the administration of the compound of Formula (I) or a pharmaceutically acceptable salt thereof. In other embodiments, where the subject was on a regimen of at least one of propranolol, primidone, topiramate, or a pharmaceutically acceptable salt thereof prior to being administered the compound of Formula (I) or a pharmaceutically acceptable salt thereof, the subject can be administered a lower dose of the propranolol, primidone, topiramate, or a pharmaceutically acceptable salt thereof than the subject was on prior to the administration of the compound of Formula (I) or a pharmaceutically acceptable salt thereof. For instance, had the subject been administered 20 mg propranolol HCl once daily prior to the administration of the compound of Formula (I) or a pharmaceutically acceptable salt thereof, the subject can be administered a dose of 10 mg propranolol HCl once daily after the subject begins administration of the compound of Formula (I) or a pharmaceutically acceptable salt thereof. In certain embodiments, the subject is administered a dose of propranolol, primidone, topiramate in combination with the compound of Formula (I) or a pharmaceutically acceptable salt thereof, wherein the dose of propranolol, primidone, topiramate is about 5% to about 90% lower than the dose of propranolol, primidone, topiramate that the subject received prior to administration the compound of Formula (I) or a pharmaceutically acceptable salt thereof. In certain embodiments, the dose of propranolol, primidone, topiramate is about 10% to about 80%, about 20% to about 70%, about 20% to about 60%, about 20% to about 50%, about 20% to about 40%, about 30% to about 60%, about 30% to about 50%, about 30% to about 40%, or about 40% to about 50%, lower than the dose of propranolol, primidone, topiramate that the subject received prior to administration the compound of Formula (I) or a pharmaceutically acceptable salt thereof. In certain embodiments, the dose of propranolol, primidone, topiramate is about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% lower than the dose of propranolol, primidone, topiramate that the subject received prior to administration the compound of Formula (I) or a pharmaceutically acceptable salt thereof.

In certain embodiments, where the subject was on a regimen of at least one of propranolol, primidone, topiramate, or a pharmaceutically acceptable salt thereof prior to being administered the compound of Formula (I) or a pharmaceutically acceptable salt thereof, the propranolol, primidone, topiramate, or a pharmaceutically acceptable salt thereof can be administered to the subject PRN (as needed). In certain embodiments, where the subject was on a regimen of propranolol or a pharmaceutically acceptable salt thereof prior to being administered the compound of Formula (I) or a pharmaceutically acceptable salt thereof, the propranolol or a pharmaceutically acceptable salt thereof can be administered to the subject PRN.

In another embodiment, the subject suffering from a disease or condition relating to aberrant function or activity of a T-type calcium channel, such as ET, who is taking at least one of propranolol, primidone, topiramate, or a pharmaceutically acceptable salt thereof (e.g., propranolol HCl) is administered a compound of Formula (I) or a pharmaceutically acceptable salt thereof (e.g., the HCl salt) on a schedule that involves titrating the dose of the compound of Formula (I) or pharmaceutically acceptable salt thereof up to a desired dosing level. For instance, in some embodiments, the dose of the compound of Formula (I) or a pharmaceutically acceptable salt thereof is titrated from a dose starting at about 5 mg daily up to a dose of about 120 mg daily. In other embodiments, the dose of the compound of Formula (I) or a pharmaceutically acceptable salt thereof is titrated from a dose starting at about 10 mg daily up to a dose of about 120 mg daily (e.g., about 20 mg daily, about 60 mg daily, about 80 mg daily, or about 100 mg daily). In other embodiments, the dose of the compound of Formula (I) or a pharmaceutically acceptable salt thereof is titrated from a dose starting at about 20 mg daily up to a dose of about 120 mg daily (e.g., titrating from about 20 mg daily in 20 mg increments to about 40 mg daily, to about 60 mg daily, to about 80 mg daily, to about 100 mg daily, or to about 120 mg daily).

In one embodiment, the subject suffering from a disease or condition relating to aberrant function or activity of a T-type calcium channel, such as ET, who is taking at least one of propranolol, primidone, topiramate, or a pharmaceutically acceptable salt thereof is administered the compound of Formula (I) or a pharmaceutically acceptable salt thereof (e.g., the HCl salt) according to a dosing regimen comprising:
(a) administering to the subject once daily for a first period (e.g., 2, 3, 4, 5, 6, 7, 8, or 9 days or more), about 5 mg per day of the compound of Formula (I) or a pharmaceutically acceptable salt thereof (e.g., the HCl salt);
(b) administering to the subject once daily for a second period (e.g., 2, 3, 4, 5, 6, 7, 8, or 9 days or more), about 10 mg per day of the compound of Formula (I) or a pharmaceutically acceptable salt thereof (e.g., the HCl salt); and
(c) administering to the subject once daily for a third period (e.g., 2, 3, 4, 5, 6, 7, 8, or 9 days or more), about 20 mg per day of the compound of Formula (I) or a pharmaceutically acceptable salt thereof (e.g., the HCl salt).

In another embodiment, the subject suffering from a disease or condition relating to aberrant function or activity of a T-type calcium channel, such as ET, who is taking at least one of propranolol, primidone, topiramate, or a pharmaceutically acceptable salt thereof is administered the compound of Formula (I) or a pharmaceutically acceptable salt thereof (e.g., the HCl salt), according to a dosing regimen comprising:
(a) administering to the subject for a first period (e.g., 2, 3, 4, 5, 6, 7, 8, or 9 days or more), about 20 mg per day of the compound of Formula (I) or a pharmaceutically acceptable salt thereof (e.g., the HCl salt);
(b) administering to the subject for a second period (e.g., 2, 3, 4, 5, 6, 7, 8, or 9 days or more), about 40 mg per day of the compound of Formula (I) or a pharmaceutically acceptable salt thereof (e.g., the HCl salt); and
(c) administering to the subject for a third period (e.g., 2, 3, 4, 5, 6, 7, 8, or 9 days or more), about 60 mg per day of the compound of Formula (I) or a pharmaceutically acceptable salt thereof (e.g., the HCl salt).

In some embodiments, the method further comprises (d) administering to the subject for a fourth period (e.g., 2, 3, 4, 5, 6, 7, 8, or 9 days or more), about 80 mg daily of the compound of Formula (I) or a pharmaceutically acceptable salt thereof (e.g., the HCl salt).

In other embodiments, the method further comprises (e) administering to the subject for a fifth period (e.g., 2, 3, 4, 5, 6, 7, 8, or 9 days or more), about 100 mg daily of the compound of Formula (I) or a pharmaceutically acceptable salt thereof (e.g., the HCl salt).

In certain embodiments, the method further comprises (f) administering to the subject for a sixth period (e.g., 2, 3, 4, 5, 6, 7, 8 or 9 days or more), about 120 mg daily of the compound of Formula (I) or a pharmaceutically acceptable salt thereof (e.g., the HCl salt).

In some embodiments, the subject is a human. In some embodiments, the subject is a human and is 10 to 100 years old, e.g., 10 to 30 years old, 15 to 45 years old, 18 to 55 years old, 10 to 80 years old, 15 to 75 years old, 40 to 90 years old, 50 to 80 years old, 60 to 75 years old or 25 to 65 years old. In some embodiments, the subject is a human and is 18 to 55 years old.

In some embodiments, the subject has ET with onset at the age of 30 years old or less, 40 years old or less, 50 years old or less, 60 years old or less, 70 years old or less or 80 years old or less. In some embodiments, the subject has ET with onset at the age of from 10 to 90 years old, from 30 to 60 years old, from 40 to 80 years old, or from 50 to 75 years old.

In some embodiments, the subject suffering from a disease or condition relating to aberrant function or activity of a T-type calcium channel, such as ET, also suffers from another disorder, including another disease or condition relating to aberrant function or activity of a T-type calcium channel. In some such embodiments, the other disorder is selected from the group consisting of psychiatric disorders (e.g., mood disorder (e.g., major depressive disorder)), pain, tremor, seizures (e.g., absence seizures), and epilepsy or an epilepsy syndrome (e.g., juvenile myoclonic epilepsy).

In some embodiments, the compound of Formula (I) or a pharmaceutically acceptable salt thereof (e.g., the HCl salt) can be administered to the subject suffering from a disease or condition relating to aberrant function or activity of a T-type calcium channel, such as ET, that has become refractory to treatment with propranolol or a pharmaceutically acceptable salt thereof. In other embodiments, the compound of Formula (I) or a pharmaceutically acceptable salt thereof (e.g., the HCl salt) can be administered to the subject suffering from a disease or condition relating to aberrant function or activity of a T-type calcium channel, such as ET, that has become refractory to other first-line therapies such as primidone and/or topiramate.

In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor severity by at least about 20% when measured by The Essential Tremor Rating Assessment Scale (TETRAS). The term “TETRAS” as used herein refers to a scale developed to quantify severity of ET and its impact on daily activities. It has an activities of daily living (ADL) section and a performance section. The ADL section has 12 items rated between 0 to 4, and the performance section has 9 items rated between 0 to 4. See, e.g., Elble, R. R., The Essential Tremor Rating Assessment Scale, J. Neurology Neuromed. 2016, 1(4):34-38.

In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor severity by at least about 30% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor severity by at least about 40% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor severity by at least about 50% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof, can reduce tremor severity by at least about 60% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor severity by from about 20% to about 70% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor severity by from about 30% to about 50% when measured by TETRAS.

In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce upper limb tremor amplitude by at least about 20% when measured by The Essential Tremor Rating Assessment Scale (TETRAS). In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce upper limb tremor amplitude by at least about 30% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce upper limb tremor amplitude by at least about 40% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof, can reduce upper limb tremor amplitude by at least about 50% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof, can reduce upper limb tremor amplitude by at least about 60% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce upper limb tremor amplitude by from about 20% to about 70% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce upper limb tremor amplitude by from about 30% to about 50% when measured by TETRAS.

In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by at least about 20% when measured by The Essential Tremor Rating Assessment Scale (TETRAS). In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by at least about 30% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by at least about 40% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by at least about 50% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by at least about 60% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by from about 20% to about 70% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude when drawing Archimedes spirals with the right or left hands by from about 30% to about 50% when measured by TETRAS.

In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by at least about 20% when measured by The Essential Tremor Rating Assessment Scale (TETRAS). In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by at least about 30% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by at least about 40% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by at least about 50% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by at least about 60% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by from about 20% to about 70% when measured by TETRAS. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can reduce tremor amplitude in handwriting by from about 30% to about 50% when measured by TETRAS.

In other embodiments, other suitable measurements to assess the efficacy of the compound of Formula (I) or a pharmaceutically acceptable salt thereof (e.g., the HCl salt), when administered in combination with at least one of propranolol, primidone, topiramate, or a pharmaceutically acceptable salt thereof include measuring the TETRAS activities of daily living (ADL) and modified activities of daily living (mADL) scores.

In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by at least about 20% when measured by TETRAS ADL. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by at least about 30% when measured by TETRAS ADL. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by at least about 40% when measured by TETRAS ADL. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by at least about 50% when measured by TETRAS ADL. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by at least about 60% when measured by TETRAS ADL. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by from about 20% to about 70% when measured by TETRAS ADL. In some embodiments, the compound of Formula (I) or a pharmaceutically acceptable salt thereof (e.g., the HCl salt), when administered in combination with at least one of propranolol, primidone, topiramate, or a pharmaceutically acceptable salt thereof can improve activities of daily living by from about 30% to about 50% when measured by TETRAS ADL.

The Clinical Global Impression-Improvement (CGI-I) (Guy 1976) assesses the clinician's impression of the participant's functioning prior to and after initiating study medication. The clinician should use his/her total clinical experience with this patient population and rate the current severity of the participant's essential tremor symptoms on a 7-point scale from 1 (Normal, not at all ill) to 7 (Among the most extremely ill patients). Accordingly, in some embodiments, the methods of the present invention result in a decrease in CGI-I score compared to baseline, e.g., prior to treatment.

The Patient Global Impression of Change (PGI-C) assesses the participant's improvement (or worsening). The participant is required to assess their condition relative to Baseline (Day 0) on a 7-point scale from 1 (very much improved) to 7 (very much worse). The assessment is made independent of whether the participant believes the improvement/worsening was drug-related or not. In some embodiments, the method results in a reduction in PGI-C score compared to baseline. The Columbia-Suicide Severity Rating Scale (C-SSRS) assesses suicidal ideation and behavior in participants during participation in a clinical trial of centrally-acting drugs. The C-SSRS is composed of 5 questions addressing suicidal behavior and 5 questions addressing suicidal ideation, with sub-questions assessing the severity. The tool should be administered via interview with the participant (by a trained operator/interviewer) and takes about 5 to 10 minutes to complete. In some embodiments, the method results in a reduction in suicidal ideation and/or behavior as compared to suicidal ideation and/or behavior prior to treatment. In other embodiments, the method results in no change in the C-SSRS score of a patient as compared to the C-SSRS score prior to treatment.

The Beck Depression Inventory (BDI-II) is a list of 21 common symptoms of depression (Beck et al 1996). Each item is scored on a 4-point Likert scale (range of scores is 0 through 3). The time frame for assessment is the preceding month or since the last assessment. The BDI-II is divided into 2 subscales: Affective and Somatic. The subscale scores are calculated as the sum of the items comprising each subscale. The total score is calculated as the sum of all 21 items and ranges from 0 to 63. A total score of 0 to 13 indicates minimal depressive symptoms. A total score of 14 to 19 indicates mild depressive symptoms. A total score of 20 to 28 indicates moderate depressive symptoms. A total score of 29 to 63 indicates severe depressive symptoms. The BDI-II is assessed at screening to exclude participants with moderate to severe depressive symptoms. Accordingly, in some embodiments, the method does not result in moderate depressive symptoms or severe depressive symptoms in the subject as measured by BDI-II.

The Beck Anxiety Index (BAI) is a brief measure of anxiety with a specific attention on somatic symptoms of anxiety that was developed as a measure aimed at discriminating between anxiety and depression (Beck et al 1988). The BAI contains 21 questions about the common symptoms of anxiety that the subject could have experienced during the past week (including the day the BAI is administered). The common symptoms of anxiety include numbness and tingling, sweating not due to heat, and fear of the worst happening. It is designed for individuals who are of 17 years of age or older and takes 5 to 10 minutes to complete. Each answer is scored on a scale value of 0 (not at all) to 3 (severely). Higher total scores indicate more severe anxiety symptoms. The standardized cutoffs are the following: 0-7: minimal, 8-15: mild, 16-25: moderate, 26-63: severe. Accordingly, in some embodiments, the method does not result in moderate anxiety symptoms or severe anxiety symptoms as measured by BAI.

### III. Dosage Forms and Compositions

In some embodiments, the compound of Formula (I) or a pharmaceutically acceptable salt thereof (e.g., the HCl salt) may be formulated in a dosage form or in a pharmaceutical composition.

In some embodiments, a composition that can be used in the methods described herein may be a pharmaceutical composition comprising the compound of Formula (I) or a pharmaceutically acceptable salt thereof and an excipient that functions to modify the release rate of the compound of Formula (I) or a pharmaceutically acceptable salt thereof. In some embodiments, the pharmaceutical composition may be a swellable core technology formulation.

In certain embodiments, a dosage form that can be used in the methods described herein may be an oral dosage form comprising: the compound of Formula (I) or a pharmaceutically acceptable salt thereof (e.g., the HCl salt); and at least one modified-release polymer. The at least one modified-release polymer includes, but is not limited to, controlled-release polymers, hydrophilic matrix polymers such as an HPMC polymer, hydrophobic matrix polymers such as ethyl cellulose and ethocel, and polyacrylate polymers such as Eudragit RL100 and Eudragit RS100. The at least one modified-release polymer is typically present in an amount sufficient to modify the release rate of the compound of Formula (I) or a pharmaceutically acceptable salt thereof (e.g., the HCl salt) upon administration to the subject.

In some embodiments, the composition or dosage form may comprise from about 0.9% by weight to about 40% by weight (e.g., from about 0.9% by weight to about 30%, from about 1% by weight to about 25% by weight, from about 2% by weight to about 25% by weight, from about 3% by weight to about 20% by weight, from about 4% by weight to about 20% by weight, from about 5% by weight to about 20% by weight, from about 5% by weight to about 15% by weight, from about 5% by weight to about 10% by weight, or about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40% by weight) of the compound of Formula (I) or a pharmaceutically acceptable salt thereof (e.g., the HCl salt). In some embodiments, the dosage form comprises about 30% by weight to about 40% by weight of the compound of Formula (I) or a pharmaceutically acceptable salt thereof (e.g., the HCl salt).

In some embodiments, the composition or dosage form may comprise from about 14% by weight to about 25% by weight of the compound of Formula (I) or a pharmaceutically acceptable salt thereof (e.g., the HCl salt). In some embodiments, the composition or dosage form comprises from about 19% by weight to about 20% by weight of the compound of Formula (I) or a pharmaceutically acceptable salt thereof (e.g., the HCl salt). In some embodiments, the composition or dosage form comprises from about 21% by weight to about 22% by weight of the compound of Formula (I) or a pharmaceutically acceptable salt thereof (e.g., the HCl salt). In some embodiments, the composition or dosage form comprises from about 4% by weight to about 15% by weight of the compound of Formula (I) or a pharmaceutically acceptable salt thereof (e.g., the HCl salt). In some embodiments, the composition or dosage form comprises from about 4% by weight to about 10% by weight of the compound of Formula (I) or a pharmaceutically acceptable salt thereof (e.g., the HCl salt). In some embodiments, the composition or dosage form comprises from about 4% by weight to about 5% by weight of the compound of Formula (I) or a pharmaceutically acceptable salt thereof (e.g., the HCl salt). In some embodiments, the composition or dosage form comprises from about 5% by weight to about 6% by weight of the compound of Formula (I) or a pharmaceutically acceptable salt thereof (e.g., the HCl salt). In some embodiments, the composition or dosage form comprises from about 9% by weight to about 10% by weight of the compound of Formula (I) or a pharmaceutically acceptable salt thereof (e.g., the HCl salt).

In other embodiments, the composition or dosage form that can be used in the methods described herein may be a dosage form or composition comprising from about 1 mg to about 40 mg (e.g., about 5 mg, about 10 mg, about 15 mg, about 20 mg, about 25 mg, about 30 mg, about 35 mg, about 40 mg) of the compound of Formula (I) or a pharmaceutically acceptable salt thereof (e.g., the HCl salt), and in some embodiments, the composition or dosage form may further comprise at least one modified-release polymer (e.g., controlled-release polymers, hydrophilic matrix polymers such as an HPMC polymer, hydrophobic matrix polymers such as ethyl cellulose and ethocel, and polyacrylate polymers such as Eudragit RL100 and Eudragit RS100). Typically, the at least one modified-release polymer is present in an amount sufficient to modify the release rate of the compound of Formula (I) or a pharmaceutically acceptable salt thereof (e.g., the HCl salt) upon administration to the subject.

In other embodiments, the composition or dosage form comprises from about 4 mg to about 6 mg (e.g., about 5 mg) of the compound of Formula (I) or a pharmaceutically acceptable salt thereof (e.g., the HCl salt). In certain embodiments, the composition or dosage form comprises from about 15 mg to about 25 mg (e.g., about 20 mg) of the compound of Formula (I) or a pharmaceutically acceptable salt thereof (e.g., the HCl salt). In some embodiments, the composition or dosage form comprises from about 5 mg to about 15 mg (e.g., about 10 mg) of the compound of Formula (I) or a pharmaceutically acceptable salt thereof (e.g., the HCl salt). In other embodiments, the composition or dosage form comprises from about 25 mg to about 35 mg (e.g., about 30 mg) of the compound of Formula (I) or a pharmaceutically acceptable salt thereof (e.g., the HCl salt). In certain embodiments, the composition or dosage form comprises from about 35 mg to about 45 mg (e.g., about 40 mg) of the compound of Formula (I) or a pharmaceutically acceptable salt thereof (e.g., the HCl salt).

In some embodiments, the composition or dosage form comprises from about 55 mg to about 65 mg of a modified-release polymer (e.g., an HPMC polymer). In some embodiments, the composition or dosage form comprises from about 10% by weight to about 70% by weight of the modified-release polymer (e.g., an HPMC polymer). In some embodiments, the composition or dosage form comprises from about 50% by weight to about 60% by weight of the modified-release polymer (e.g., an HPMC polymer).

In some embodiments, the composition or dosage form further comprises at least one diluent. In some embodiments, the diluent comprises microcrystalline cellulose. In some embodiments, the composition or dosage form comprises from about 15 mg to about 40 mg (e.g., from about 15 mg to about 25 mg, from about 20 mg to about 25 mg, from about 25 mg to about 30 mg, or from about 30 mg to about 40 mg) microcrystalline cellulose. In some embodiments, the composition or dosage form comprises from about 15 mg to about 25 mg microcrystalline cellulose. In some embodiments, the composition or dosage form comprises from about 30 mg to about 40 mg microcrystalline cellulose. In some embodiments, the composition or dosage form comprises from about 15% to about 35% by weight (e.g., from about 15% to about 20%, from about 20% to about 25%, from about 25% to about 30%, or from about 30% to about 35% by weight) microcrystalline cellulose.

In some embodiments, the composition or dosage form further comprises at least one glidant. In some embodiments, the glidant comprises colloidal silicon dioxide. In some embodiments, the composition or dosage form further comprises at least one lubricant. In some embodiments, the lubricant comprises magnesium stearate. In some embodiments, the composition or dosage form further comprises at least one coating.

In some embodiments, about 80% of the compound of Formula (I) or a pharmaceutically acceptable salt thereof (e.g., the HCl salt) is released within 7 hours upon administration to a subject. In certain embodiments, about 80% of the compound of Formula (I) or a pharmaceutically acceptable salt thereof (e.g., the HCl salt) is released within 7 hours, as measured by using USP apparatus type-I, media containing 900 mL 0.1 M HCl, and a paddle speed of 100 rpm.

In some embodiments, the composition or dosage form, upon administration to a subject, has a reduced Cₘₐₓ value as compared to a reference oral dosage form (e.g., a composition or dosage form with any intended release rate profile, including, for example, a modified release rate profile, a composition or dosage form that does not have a modified release rate profile, or a composition or dosage form that does not have a modified-release polymer, e.g., an HPMC polymer). In some embodiments, the composition or dosage form, upon administration to a subject, has a greater tₘₐₓ value than a reference oral composition or dosage form (e.g., a composition or dosage form with any intended release rate profile, including, for example, a modified release rate profile, a composition or dosage form that does not have a modified release rate profile, or a composition or dosage form that does not have a modified-release polymer, e.g., an HPMC polymer).

In other embodiments, the composition or dosage form is administered to a patient once daily. In certain embodiments, the composition or dosage form is administered to a patient twice daily. In some embodiments, the dosage form is a tablet. In other embodiments, the dosage form is a capsule. In certain embodiments, the dosage form is a suspension.

In some embodiments, a dosage form that can be used in the methods described herein may be an oral dosage form comprising: from about 15 mg to 25 mg of the compound of Formula (I) or a pharmaceutically acceptable salt thereof (e.g., the HCl salt); and from about 55 mg to 65 mg of an HPMC polymer.

In other embodiments, a dosage form that can be used in the methods described herein may be an oral dosage form comprising: from about 14% by weight to about 25% by weight of the compound of Formula (I) or a pharmaceutically acceptable salt thereof (e.g., the HCl salt); and from about 53% to about 64% by weight of an HPMC polymer.

In certain embodiments, a dosage form that can be used in the methods described herein may be an oral dosage form comprising: from about 3 mg to 8 mg of the compound of Formula (I) or a pharmaceutically acceptable salt thereof (e.g., the HCl salt); and from about 55 mg to 65 mg of an HPMC polymer.

In some embodiments, a dosage form that can be used in the methods described herein may be an oral dosage form comprising: from about 3% by weight to about 8% by weight of the compound of Formula (I) or a pharmaceutically acceptable salt thereof (e.g., the HCl salt); and from about 53% to about 64% by weight of an HPMC polymer.

In other embodiments, a dosage form that can be used in a method described herein may be an oral (e.g., particulate) composition comprising: the compound of Formula (I) or a pharmaceutically acceptable salt thereof (e.g., the HCl salt); and a modified-release polymer (e.g., a controlled-release polymer such as an HPMC polymer or a hydrophilic matrix polymer).

In some embodiments, the compound of Formula (I) or a pharmaceutically acceptable salt thereof (e.g., the HCl salt) is stable within the composition at about 25° C. at 60% relative humidity for at least 24 months. In some embodiments, the compound of Formula (I) or a pharmaceutically acceptable salt thereof (e.g., the HCl salt) is stable within the composition at about 25° C. at 60% relative humidity for at least 36 months. In some embodiments, the compound of Formula (I) or a pharmaceutically acceptable salt thereof (e.g., the HCl salt) is stable with the composition at about 25° C. at 60% relative humidity for at least 48 months. In other embodiments, the compound of Formula (I) or a pharmaceutically acceptable salt thereof (e.g., the HCl salt) is stable within the composition at about 25° C. at 60% relative humidity for at least 60 months. In some embodiments, the compound of Formula (I) or a pharmaceutically acceptable salt thereof (e.g., the HCl salt) is stable within the composition at about 40° C. at 75% relative humidity for at least 6 months.

In another aspect, provided herein is an oral (e.g., particulate) composition comprising: from about 15 mg to about 25 mg of the compound of Formula (I) or a pharmaceutically acceptable salt thereof (e.g., the HCl salt); and from about 55 mg to about 65 mg HPMC.

In other embodiments, a composition that can be used in the methods described herein may be an oral (e.g., particulate) composition comprising: from about 14% by weight to about 25% by weight of the compound of Formula (I) or a pharmaceutically acceptable salt thereof (e.g., the HCl salt); and from about 53% to about 64% by weight of an HPMC polymer.

In certain embodiments, a composition that can be used in the methods described herein may be an oral (e.g., particulate) composition comprising: from about 3 mg to about 8 mg of the compound of Formula (I) or a pharmaceutically acceptable salt thereof (e.g., the HCl salt); and from about 55 mg to about 65 mg HPMC.

In some embodiments, a composition that can be used in the methods described herein may be an oral (e.g., particulate) composition comprising: from about 3% by weight to about 8% by weight of the compound of Formula (I) or a pharmaceutically acceptable salt thereof (e.g., the HCl salt); and from about 53% to about 64% by weight of an HPMC polymer.

In another aspect, the disclosure provides a fixed-dose oral composition comprising the compound of Formula (I) or a pharmaceutically acceptable salt thereof (e.g., the HCl salt) and at least one of propranolol, primidone, topiramate, or a pharmaceutically acceptable salt thereof (e.g., propranolol HCl). In certain embodiments, the fixed-dose composition may be formulated for once daily administration. In some variations, the propranolol in the fixed-dose composition is (S)-propranolol, (R)-propranolol, or a mixture of (S)- and (R)-propranolol. In certain embodiments, the propranolol in the fixed-dose composition is (S)-propranolol HCl. In certain embodiments, the propranolol in the fixed-dose composition is (R)-propranolol HCl. In yet other embodiments, the propranolol in the fixed-dose composition is a mixture of (S)- and (R)-propranolol HCl. In certain embodiments, the fixed-dose composition includes at least one of propranolol, primidone, topiramate, or a pharmaceutically acceptable salt thereof, and the compound of Formula (I) or a pharmaceutically acceptable salt thereof, present in a ratio of from about 1:10 to about 10:1 by weight in the composition. For instance, the ratio between the at least one of propranolol, primidone, topiramate, or a pharmaceutically acceptable salt thereof, and the compound of Formula (I) or a pharmaceutically acceptable salt thereof, can be about 1:10, 1:5, 1:4, 1:3, 1:2, 1:1, 2:1, 3:1, 4:1, 5:1 or 10:1 by weight. It will be understood that all weights are based on the weight of the free base of the compound of Formula (I) and the at least one of propranolol, primidone, or topiramate.

### Immediate-Release Formulations

In some embodiments, a dosage form or composition that can be used in the methods described herein may be a dosage form or composition comprising the compound of Formula (I) or a pharmaceutically acceptable salt thereof (e.g., the HCl salt), where the compound of Formula (I) or a pharmaceutically acceptable salt thereof (e.g., the HCl salt) is released immediately upon an administration to the subject.

In other embodiments, a dosage form that can be used in the methods described herein may be an oral capsule for immediate release comprising: from about 15 mg to about 20 mg of the compound of Formula (I) or a pharmaceutically acceptable salt thereof (e.g., the HCl salt); and from about 75 mg to about 85 mg of at least one diluent; from about 2 mg to about 10 mg of at least one binder; from about 1% to about 5% of at least one disintegrant; and from about 0.1 mg to about 5 mg of at least one lubricant.

### Administrations

In some embodiments, the dosage form or composition is administered to the subject more than once a day (e.g., twice a day, three times a day, or four times a day). In some embodiments, the compound of Formula (I) or a pharmaceutically acceptable salt thereof and at least one of propranolol, primidone, topiramate or a pharmaceutically acceptable salt thereof are administered in separate pharmaceutical compositions. In certain embodiments, each composition is administered orally once daily to the subject in need thereof.

In some embodiments, the dosage form or composition is administered to the subject once a day (e.g., one 20 mg tablet per day, two 20 mg tablets a day, or three 20 mg tablets a day). In some embodiments, the dosage form or composition is administered to the subject twice a day. In some embodiments, the dosage form or composition is administered to the subject every other day. In certain embodiments, about 1 mg to about 40 mg of the compound of Formula (I) or a pharmaceutically acceptable salt thereof (e.g., the HCl salt) is administered to the subject daily. In other embodiments, about 15 mg to about 25 mg of the compound of Formula (I) or a pharmaceutically acceptable salt thereof (e.g., the HCl salt) is administered to the subject daily. In certain embodiments, about 30 mg to about 40 mg of the compound of Formula (I) or a pharmaceutically acceptable salt thereof (e.g., the HCl salt) is administered to the subject daily.

### EXAMPLES

In order that the embodiments described herein may be more fully understood, the following examples are set forth. The examples described in this application are offered to illustrate the compounds, pharmaceutical compositions, and methods provided herein and are not to be construed in any way as limiting their scope.

### Example 1: A Phase 2 Clinical Trial Evaluating the Efficacy, Safety, Tolerability, and Pharmacokinetics of a Compound of Formula (I) in Adults with Essential Tremor

This multi-center clinical trial assessed the efficacy, safety, tolerability, and PK of the HCl salt compound of Formula (I) in participants aged 18 years of age or older who have had signs and symptoms consistent with ET for at least 3 years, with an onset before age 65. The clinical trial was conducted in 2 parts (Part A and Part B). Both parts consisted of 3 periods: Screening/Baseline, Intervention, and Safety Follow-up Periods.

Part A of the clinical trial was open-label and assessed the safety and tolerability of the study drug, as well as the overall magnitude and pattern of change in ET severity. Daily dose levels of the HCl salt compound of Formula (I) were titrated from 20 mg (Day 1 to Day 7) to 40 mg (Days 8 to 14).

Part B of the clinical trial consisted of both an open-label titration phase and a randomized, double-blind, placebo-controlled withdrawal phase. Part B assessed the safety and tolerability of the study drug, as well as the overall magnitude and pattern of change in ET severity and the duration of that effect. During the open-label titration phase, the daily dose levels of the HCl salt compound of Formula (I) were titrated from 20 mg (Days 1 to 3) to 40 mg (Days 4 to 7) to 60 mg (Days 8 to 14) to 80 mg (Days 15 to 21) to 100 mg (Days 22 to 28) to 120 mg (Days 29 to 42) or the highest tolerated dose. In the randomized, double-blind, placebo-controlled withdrawal phase, participants were either maintained on their final open-label dose or switched to placebo for an additional 14 days (Days 43 to 56).

Participants who received study drug in Part A were not eligible for Part B.

### Screening/Baseline Period

The Screening period for Part A and Part B lasted up to 28 days (Day −28 to Day −1). Fourteen additional days (i.e., 42 days total) were allowed in the Screening period for participants who discontinued primidone.

Key Screening assessments included medical history, demographics, physical examination, drug screen, clinical laboratory evaluations and serum pregnancy for women of childbearing potential, 12-lead ECG, vital signs, C-SSRS, assessment of ET severity using the TETRAS Performance subscale (including a video for independent review of eligibility), and a review of concomitant medications. To be eligible, potential participants who were taking prohibited medications needed to successfully discontinue those medications for at least 5 half-lives or 14 days prior (whichever was the longer period of time) before the first dose of study drug.

For those participants requiring the 14 additional days in the Screening period to discontinue primidone, all or some of the Screening assessments may need to be repeated.

For Part A, participants completed Baseline assessments (Day 0). Day 0 (Baseline) activities were combined with Day 1 activities, provided that Baseline activities were completed before dosing and that dosing occurred in the morning of Day 1. In addition, Baseline efficacy assessments were either conducted on Day 0 or Day 1 pre-dose, but not both to avoid learning effects.

For participants in Part B, TETRAS Upper Limb items were also completed with accelerometry at Screening and via telehealth on Day −2 of the Screening period. The Screening telehealth visit was needed to ensure participants could complete the TETRAS Upper Limb assessment successfully via telehealth and to provide a pre-dose measure. Additional assessments added to Part B screening included TETRAS ADL and a Clinical Global Impressions scale measuring severity of illness (CGI-S).

### Intervention Period

Part A. On Day 1, participants received a 20 mg dose of study drug, administered in the morning. Participants remained in a clinical setting under medical observation for approximately 6 hours. TETRAS Performance assessments were performed before dosing and 6 hours post-dose. After the first dose, participants continued QAM dosing at home after breakfast through Day 7. Participants returned to the clinic on Day 7 for efficacy testing at the 20 mg dose level. On Day 8, the dose increased to 40 mg QAM after breakfast through Day 14. Participants returned to the clinic on Day 14 for efficacy testing at the 40 mg dose level. Participants who agreed to optional additional PK sampling remained overnight in the clinic on Day 1, Day 7, and Day 14.

Key safety measures included clinical laboratory evaluations, 12-lead ECG, C-SSRS, and vital signs. Key efficacy assessments included the TETRAS Performance subscale, TETRAS Upper Limb using accelerometry, CGI, and PGI-C. Blood samples were obtained for the determination of study drug plasma concentrations using a validated bioanalytical method and may also be used for method development and/or metabolite characterization.

### Part B. Open-Label Titration Phase

On Day 1, participants received a 20 mg dose of study drug on an empty stomach (at least 1 hour before breakfast). Participants remained in a clinical setting under medical observation for approximately 6 hours. TETRAS Performance assessments were performed before dosing and 6 hours (±2) post-dose. For dosing on Day 2 through Day 41, participants were instructed to dose QAM at least 1 hour before breakfast at home. Participants continued taking study drug 20 mg QAM at home through Day 3. On Day 3, participants received a telephone call from site staff to inquire about adverse events (AEs) and concomitant medications, confirm dose escalation, and remind the participant about the number of study drug tablets to take on Day 4 through Day 7. On Day 4, participants escalated to 40 mg QAM through Day 7, when they returned to the clinic for safety assessments and efficacy testing. On Day 8, participants escalated to 60 mg QAM through Day 14. On Day 14, participants received a telephone call from site staff to inquire about AEs and concomitant medications, confirm dose escalation, and remind the participant about the number of study drug tablets to take on Day 15 through Day 21. On Day 15, participants escalated to 80 mg QAM through Day 21, when they returned to the clinic for safety assessments and efficacy testing. On Day 22, participants escalated to 100 mg QAM through Day 28. On Day 28, participants received a telephone call from site staff to inquire about AEs and concomitant medications, confirm dose escalation, and remind the participant about the number of study drug tablets to take on Day 29 through Day 42. On Day 29, participants escalated to 120 mg QAM through Day 42, when they returned to the clinic for safety assessments and efficacy testing. The dose on Day 42 was taken in the clinic. On Day 35, participants received a telephone call from site staff to inquire about AEs and concomitant medications. On Day 41, participants completed a TETRAS Performance Upper Limb assessment via a telehealth visit. If at any point during the open-label titration phase a participant did not tolerate escalation and the participant was returned to a lower dose level, the participant could continue according to the schedule outlined above, but no further dose changes were allowed. Participants could only change to a lower dose level once, and no dose changes were allowed to occur after Day 36.

### Double-Blind Randomized Withdrawal Phase

On Day 42, participants completed the final clinic visit of the open-label titration phase. Participants received their dose of study drug in the clinic on an empty stomach (at least 1 hour before breakfast). Participants remained in a clinical setting under medical observation for approximately 6 hours. TETRAS Performance assessments were performed before dosing and 6 hours (±2) post-dose.

After Day 42, participants either continued receiving study drug at their current dose or switched to placebo, depending on their randomization. Randomization in a 1:1 fashion was completed at any time on Day 37 through 42.

Participants were instructed to take their assigned blinded treatment on Day 43 through Day 56 QAM (at least 1 hour before breakfast). On Day 56 (the last day of dosing), participants returned to the clinic for safety assessments and efficacy testing.

In both phases, key safety measures included clinical laboratory evaluations, 12-lead ECG, C-SSRS, and vital signs. Key efficacy assessments included the TETRAS Performance subscale, TETRAS Upper Limb using accelerometry, CGI, PGI-C, TETRAS ADL Subscale, QUEST, BDI-II, and BAI. Blood samples were obtained for the determination of study drug and metabolite plasma concentrations.

### Safety Follow-Up Period

Part A. The Safety Follow-up period took place from Day 15 to Day 21. At the end of the Safety Follow-up period, participants returned to the clinic on Day 21 (±1 day) for the final clinical trial assessments.

Part B. The Safety Follow-up period took place from Day 57 to Day 70. At the end of the Safety Follow-up period, participants returned to the clinic on Day 70 for the final clinical trial assessments.

### Number of Participants

Part A. In Part A of the study, 7 participants were administered study drug.

Part B. In Part B of the study, 17 participants were administered study drug for the first 42 days followed by randomization (1:1) for 2 additional weeks on either the highest tolerated dose of study drug or placebo.

### Duration of Clinical Trial

Participants were part of the clinical trial for the following time intervals, set forth in Tables 1 and 2 below.

TABLE 1
Part A

| | |
|---|---|
| Screening/Baseline Period | Up to 20 days (+14 days if on primidone) |
| Intervention Period | 14 days |
| Safety Follow-up Period | 7 days |
| Total Participation | Up to 49 days (63 days if on primidone) |

TABLE 2
Part B

| | |
|---|---|
| Screening/Baseline Period | Up to 28 days (+14 days if on primidone) |
| Intervention Period | 56 days |
| Safety Follow-up Period | 14 days |
| Total Participation | Up to 98 days (112 days if on primidone) |

### Test Product, Reference Therapy, and Administration

20 mg of the HCl salt compound of Formula (I) MR7 tablets or matching placebo were administered orally and provided in pre-packaged containers to the participants.

### Dose/Route/Regiment

Part A. All participants received study drug orally, 20 mg QAM for 7 days (Day 1 through Day 7) and 40 mg QAM for 7 days (Day 8 through Day 14).

Part B. Open-Label Titration The titration schedule for study drug was as follows:
20 mg QAM for 3 days (Day 1 through Day 3);
40 mg QAM for 4 days (Day 4 through Day 7);
60 mg QAM for 7 days (Day 8 through Day 14);
80 mg QAM for 7 days (Day 15 through Day 21);
100 mg QAM for 7 days (Day 22 through Day 28); and
120 mg QAM for 14 days (Day 29 through Day 42).

Double-Blind Randomized Withdrawal. Participants were randomized to either continue receiving study drug orally QAM at their highest tolerated dose or switch to receiving placebo orally QAM for 14 days (Day 43 through Day 56).

### Objectives and Endpoints

To evaluate the efficacy, safety, tolerability, and pharmacokinetics (PK) of study drug in adults with ET.

TABLE 3
Part A Objectives and Endpoints

| | |
|---|---|
| Objective | Endpoint |

Primary

| | |
|---|---|
| To evaluate the efficacy of study drug on upper | Change from Baseline to Day 7 and Day 14 in |
| limb tremor in participants with ET | The Essential Tremor Rating Assessment Scale |
| | (TETRAS) upper limb score |

Secondary

| | |
|---|---|
| To evaluate the efficacy of study drug on | Change from Baseline to Day 7 and Day 14 in: |
| other measures of tremor severity in | TETRAS Performance subscale total score |
| participants with ET | Accelerometer-based upper limb score |
| | TETRAS Performance subscale individual |
| | item scores |
| | TETRAS CUL score |
| To evaluate the safety and tolerability of | Incidence and severity of adverse events (AEs) |
| study drug in participants with ET | Changes in vital sign measurements |
| | Changes in clinical laboratory results |
| | Changes in electrocardiogram (ECG) |
| | parameters |
| | Incidence of Columbia-Suicide Severity Rating |
| | Scale (C-SSRS) measured suicidal ideation or |
| | behavior |

Other

| | |
|---|---|
| To evaluate the effects of study drug on | Change from Baseline to Day 7 and Day 14 in: |
| central ratings of tremor severity | TETRAS Performance subscale total score, |
| using central video ratings | upper limb scores, and individual item scores |
| | determined via independent rater review of |
| | videos |
| To evaluate the effects of study drug on | Change from Baseline to Day 7 and Day 14 in: |
| central ratings of tremor severity using | Clinical Global Impression of Severity |
| global measures of disease state | (CGI-S) scores |
| | Patient Global Impression of Change (PGI-C) |
| | scores at Day 7 and Day 14 |
| | Clinical Global Impression of Improvement |
| | (CGI-I) scores on Day 7 and Day 14 |
| To determine plasma concentrations of study | Plasma study drug and metabolite |
| drug and its metabolites following multiple | concentrations over time |
| dose administration in a titration regimen | |
| To characterize the pharmacokinetic | Primary PK parameters: Cₘₐₓ, Tₘₐₓ, AUC₀₋ₜₐᵤ, |
| (PK) profile of study drug | CL/F, Vd/F, and t_{1/2} if feasible |
| To characterize the effects of study | Screening, Baseline, Day 7, Day 14, and Day |
| drug on urine biomarkers | 21 urinary biomarkers including kidney injury |
| | molecule-1 (KIM-1) concentration in the urine |
| To evaluate the relationship between | Relationship between PK parameters, and |
| exposure to study drug and clinical | change in TETRAS Performance scores |
| measures of ET severity | |

TABLE 4
Part B Objectives and Endpoints

| | |
|---|---|
| Objective | Endpoint |

Primary

| | |
|---|---|
| To evaluate the safety and tolerability of | Incidence and severity of adverse events (AEs) |
| study drug in participants with ET | Changes in vital sign measurements |
| | Changes in clinical laboratory results |
| | Changes in electrocardiogram (ECG) parameters |
| | Incidence of Columbia-Suicide Severity Rating |
| | Scale (C-SSRS) measured suicidal ideation or |
| | behavior |

Secondary

| | |
|---|---|
| To evaluate the efficacy of study drug on | Change from Baseline to Day 42 in TETRAS |
| upper limb tremor in participants with ET | Upper Limb score |
| To evaluate the efficacy of study drug on | Change from Baseline to Day 7 and Day 21 in |
| other measures of tremor severity in | TETRAS Upper Limb score (bilateral sum of |
| participants with ET | items 4a, 4b, and 4c) |
| | Change from Baseline to Day 7, Day 21, and |
| | Day 42 in the following: |
| | TETRAS Performance subscale total score |
| | Accelerometer-based upper limb score |
| | TETRAS Performance individual item scores |
| To evaluate the efficacy of study drug | Change from Baseline to Day 7, Day 21, and |
| on measures of disease impact in | Day 42 in the following: |
| participants with ET | TETRAS ADL subscale score |
| | Quality of Life in Essential Tremor |
| | Questionnaire (QUEST) total and subscale scores |

Other

| | |
|---|---|
| To evaluate the effects of study drug on | Change from Baseline to Day 7, Day 21, and |
| tremor severity using central video ratings | Day 42 in TETRAS Performance subscale total |
| | score, upper limb scores, and individual item |
| | scores determined via independent rater review |
| | of videos |
| To evaluate the effects of study drug on upper | Change from Day −2 to Day 41 in telehealth |
| limb tremor using telehealth measures | TETRAS Upper Limb score |
| To evaluate the effects of study drug on global | Change from Baseline to Day 7, Day 21, and |
| measures of disease state | Day 42 in CGI-S score |
| | PGI-C and CGI-I scores at Day 7, Day 21, and |
| | Day 42 |
| To evaluate the effects of study drug on | Change from Baseline to Day 7, Day 21, and |
| measures of mood in participants with ET | Day 42 in the following |
| | Beck Depression Inventory - Second Edition |
| | (BDI-II) total and subscale scores |
| | Beck Anxiety Inventory (BAI) total score |
| To evaluate the duration of study drug | Change from Day 42 to Day 56 and Day 70 on |
| effect on upper limb tremor, other | the following measures: TETRAS Upper Limb score |
| measures of tremor severity, measures of | TETRAS Performance subscale total score (as |
| disease impact, mood, and global | determined by investigator and central rating) |
| measures of disease state in | Accelerometer-based upper limb score |
| participants with ET | TETRAS Performance subscale individual |
| | item scores |
| | TETRAS ADL subscale score |
| | QUEST total and subscale scores |
| | BDI-II total and subscale scores |
| | BAI total score |
| | CGI-S score |
| | PGI-C and CGI-I scores at Day 56 and 70 |
| To determine plasma concentrations | Plasma study drug and metabolite |
| of study drug and its metabolites | concentrations over time |
| following multiple dose administration | |
| in a titration regimen | |

### Results and Observations

The following table provides the study demographics representative of the ET population:

TABLE 5

| Baseline | Part A | | Part B | |
|---|---|---|---|---|
| demographics | (N = 7) | | (N = 17) | |
| Age, mean (SD) | 67.7 | (6.60) | 59.3 | (13.81) |
| Disease Duration | 42.08 | | 27.96 | |
| (years), mean | | | | |
| Gender (Male/Female) | 5/2 | (71%/29%) | 14/3 | (82%/18%) |
| (n, %) | | | | |
| # presently on | 6 | (86%) | 5 | (29%) |
| propranolol (n, %) | | | | |
| # previously on ET | 3 | (43%) | 12 | (71%) |
| medication (n, %) | | | | |
| Family History - First-degree | 3 | (43%) | 10 | (59%) |

relative with ET (n, %)

Referring to the Table 5 above, 6 participants in Part A were on propranolol, and 5 participants in Part B were on propranolol.

TABLE 6
Part A Baseline and Change from Baseline in TETRAS Efficacy Endpoints
Following Administration of Study Drug.

| | | | | | | Day 21 | |
|---|---|---|---|---|---|---|---|
| | Pre-Dose | Day 7 | | Day 14 | | (FU Assessment) | |
| | Baseline | N = 6 | | N = 6 | | N = 6 | |
| | N = 7 | Score CFB^{a} | | Score CFB^{a} | | Score CFB^{a} | |
| | Score | LS Mean | CTA^{a} | LS Mean | CTA^{a} | LS Mean | CTA^{a} |
| | Mean | (95% CI) | LS | (95% CI) | LS | (95% CI)\| | LS |
| TETRAS | (SD) | [p-value] | Mean % | [p-value] | Mean % | [p-value] | Mean % |
| ULS | 12.36 | −1.427 | −23.9% | −2.912 | −42.8% | −1.183 | −20.3% |
| | (1.973) | (−4.4375, 1.5845) | | (−4.2779, −1.5466) | | (−2.7734, 0.4064) | |
| | | [0.1048] | | [0.0028] | | [0.1129] | |
| CULS | 22.21 | −1.667 | −16.0% | −4.239 | −35.8% | −0.278 | −2.9% |
| | (4.545) | (−5.8826, 2.5476) | | (−8.3234, −0.1546) | | (−4.0192, 3.4625) | |
| | | [0.3594] | | [0.0444] | | [0.8546] | |
| PSTS | 26.29 | −1.990 | −13.3% | −5.163 | −31.0% | −1.293 | −8.9% |
| | (4.846) | (−7.5467, 3.5672) | | (−11.1371, 0.8108) | | (−6.6037, 4.0173) | |
| | | [0.4025] | | [0.0766] | | [0.5571] | |
| PS Item 6: | 2.14 | −0.091 | −9.9% | −0.346 | −32.9% | −0.061 | −6.7% |
| Archimedes | (0.900) | (−0.5713, 0.389) | | (−0.9765, 0.2836) | | (−1.0588, 0.9376) | |
| Spiral (Right) | | [0.4722] | | [0.2050] | | [0.8558] | |
| PS Item 6: | 2.57 | −0.213 | −21.8% | −0.468 | −41.7% | 0.201 | 20.7% |
| Archimedes | (1.134) | (−1.2762, 0.8501) | | (−1.3845, 0.4487) | | (−0.5882, 0.9908) | |
| Spiral (Left) | | [0.6215] | | [0.2364] | | [0.5425] | |
| PS Item 7: | 1.29 | −0.012 | −1.4% | −0.348 | −33.0% | 0.330 | 31.6% |
| Handwriting | (1.113) | (−0.7215, 0.6975) | | (−1.0966, 0.4013) | | (−0.6965, 1.3573) | |
| | | [0.9648] | | [0.2855] | | [0.4468] | |

CFB = change from pre-dose baseline; CI = confidence interval; CTA = change in tremor amplitude (from pre-dose baseline); CULS = combined upper limb score; FAS = full analysis set; LS = least square; PS = performance subscale; PSTS = performance subscale total score; TETRAS = The Essential Tremor Rating Assessment Scale; ULS = upper limb score.
Note:
Reported p-values tested the LS mean for each timepoint versus zero.
^{a}Change from pre-dose baseline.

TABLE 7
Part B Baseline and Change from Baseline in TETRAS Efficacy Endpoints Following Administration of Study Drug.

| | | Day 7 | | Day 21 | | Day 42^{a} | |
|---|---|---|---|---|---|---|---|
| | | N = 10 | | N = 12 | | N = 11 | |
| | Baseline | Score | | Score | | Score | |
| | N = 17 | CFB^{b} | | CFB^{b} | | CFB^{b} | |
| | Score | LS Mean | CTA^{b} | LS Mean | CTA^{b} | LS Mean | CTA^{b} |
| | Mean | (95% CI) | LS | (95% CI) | LS | (95% CI) | LS |
| TETRAS | (SD) | [p-value] | Mean % | [p-value] | Mean % | [p-value] | Mean % |
| ULS | 10.94 | −0.967 | −16.9% | −1.143 | −19.7% | −2.090 | −33.0% |
| | (2.397) | (−2.1795, 0.2447) | | (−2.1269, −0.1598) | | (−3.8223, −0.3585) | |
| | | [0.1042] | | [0.0257] | | [0.0216] | |
| CULS | 20.74 | −0.909 | −9.1% | 1.320 | −12.9% | −1.882 | −17.9% |
| | (4.750) | (−2.9414, 1.1234) | | (−2.8986, 0.2589) | | (−4.8650, 1.1014) | |
| | | [0.3292] | | [0.0932] | | [0.1921] | |
| PSTS | 24.06 | −1.253 | −8.6% | −1.449 | −9.9% | −2.771 | −18.1% |
| | (6.901) | (−3.8185, 1.3133) | | (−3.7708, 0.8734) | | (−6.2554, 0.7127) | |
| | | [0.2971] | | [0.2034] | | [0.1105] | |
| PS Item 6: | 2.06 | 0.006 | 0.6% | −0.240 | −24.2% | −0.239 | −24.1% |
| Archimedes | (0.682) | (−0.4387, 0.4500) | | (−0.5006, 0.0202) | | (−0.6711, 0.1930) | |
| Spiral (Right) | | [0.9776] | | [0.0668] | | [0.2460] | |
| PS Item 6: | 2.03 | 0.221 | 22.4% | −0.012 | −1.3% | −0.072 | −8.0% |
| Archimedes | (0.695) | (−0.1329, 0.5739) | | (−0.2547, 0.2316) | | (−0.4488, 0.3044) | |
| Spiral (Left) | | [0.1962] | | [0.9191] | | [0.6736] | |
| PS Item 7: | 1.65 | −0.019 | −2.2% | 0.107 | 11.5% | −0.054 | −6.0% |
| Handwriting | (1.169) | (−0.6477, 0.6087) | | (−0.3613, 0.5745) | | (−0.6984, 0.5911) | |
| | | [0.9419] | | [0.6075] | | [0.8511] | |
| mADLc | 16.35 | −2.862 | −18.49% | −2.812 | −14.31% | −6.669 | −42.04% |
| (Part B | (3.968) | (−4.9674, −0.7571) | | (−5.7299, 0.1065) | | (−10.0333, −3.3042) | |
| Only) | | [0.0126] | | [0.0575] | | [0.0013] | |
| ADL | 26.41 | −1.986 | −7.29% | −3.475 | −12.02% | −8.207 | −29.59% |
| (Part B | (3.163) | (−4.8142, 0.8417) | | (−7.0260, 0.0756) | | (−12.5655, −3.8481) | |
| Only) | | [0.1505] | | [0.0543] | | [0.0015] | |

CFB = change from baseline; CI = confidence interval; CTA = change in tremor amplitude (from pre-dose baseline); CULS = combined upper limb score; FAS = full analysis set; LS = least square; mADL = modified activities of daily living; OLT = open-label titration; PS = performance subscale; PSTS = performance subscale total score; TETRAS = The Essential Tremor Rating Assessment Scale; ULS = upper limb score.
Note:
Reported p-values tested the LS mean for each timepoint versus zero.
^{a}Change from baseline to Day 42, 6 hours post-dose.
^{b}Change from pre-dose baseline.
cmADL is referred to as TETRAS modified total score

TABLE 8
Part B Baseline and Change from Baseline in TETRAS Efficacy
Endpoints Following Randomized Withdrawal of Study Drug

| | Randomization Baseline^{a} | | Day 56 | | Day 70 | |
|---|---|---|---|---|---|---|
| | Placebo | Study drug | Placebo | Study drug | Placebo | Study drug |
| TETRAS | N = 5 | N = 6 | N = 5 | N = 6 | N = 5 | N = 6 |
| Parameter | Mean (SD) | | LS Mean CFB (95% CI) [p-value] CTA (%)^{b} | | | |
| ULS | 10.00 | 10.25 | 0.969 | 0.716 | 1.969 | 1.653 |
| | (3.317) | (1.084) | (−2.4752, | (−2.4290, | (−0.2731, | (−0.4283, |
| | | | 4.4139) | 3.8619) | 4.2118) | 3.7347) |
| | | | [0.5180] | [0.5987] | [0.0766] | [0.1042] |
| | | | 17.0% | 12.8% | 31.5% | 27.2% |
| LS Mean Difference: Study drug - | | | −0.253 (−4.9188, 4.4129) | | −0.316 (−3.3765, 2.7441) | |
| Placebo (95% CI) | | | | | | |
| p-value: Treatment Group LS Mean | | | 0.8992 | | 0.8160 | |
| Difference | | | | | | |
| CULS | 19.30 | 19.33 | 3.420 | 1.525 | 4.120 | 3.156 |
| | (8.020) | (3.157) | (−1.6327, | (−3.0887, | (−0.0017, | (−0.6797, |
| | | | 8.4732) | 6.1378) | 8.2422) | 6.9924) |
| | | | [0.1574] | [0.4683] | [0.0501] | [0.0950] |
| | | | 30.1% | 14.7% | 35.0% | 28.1% |
| LS Mean Difference: Study drug - | | | −1.896 (−8.7365, 4.9451) | | −0.964 (−6.5938, 4.6660) | |
| Placebo (95% CI) | | | | | | |
| p-value: Treatment Group LS Mean | | | 0.5410 | | 0.7042 | |
| Difference | | | | | | |
| PSTS | 22.70 | 22.83 | 4.199 | 1.174 | 4.699 | 3.422 |
| | (11.601) | (4.389) | (−1.8453, | (−4.3436, | (0.1426, | (−0.8603, |
| | | | 10.2434) | 6.6922) | 9.2556) | 7.7040) |
| | | | [0.1489] | [0.6387] | [0.0447] | [0.1034] |
| | | | 26.1% | 8.1% | 28.7% | 21.8% |
| LS Mean Difference: Study drug - | | | −3.025 (−11.2090, 5.1595) | | −1.277 (−7.5286, 4.9741) | |
| Placebo (95% CI) | | | | | | |
| p-value: Treatment Group LS Mean | | | 0.4212 | | 0.6509 | |
| Difference | | | | | | |
| PS Item 6: | 1.90 | 1.75 (0.247) | 0.497 | 0.087 | 0.297 | 0.353 |
| Archimedes | (0.742) | | (−0.1860, | (−0.5387, | (−0.2626, | (−0.1779, |
| Spiral (Right) | | | 1.1806) | 0.7120) | 0.8573) | 0.8832) |
| | | | [0.1331] | [0.7596] | [0.2532] | [0.1645] |
| | | | 43.6% | 9.5% | 29.0% | 33.4% |
| PS Item 6: | 2.20 | 1.75 (0.524) | 0.423 | 0.394 | 0.223 | 0.384 |
| Archimedes | (1.095) | | (−0.0700, | (−0.0575, | (−0.4212, | (−0.2676, |
| Spiral (Left) | | | 0.9153) | 0.8451) | 0.8665) | 1.0365) |
| | | | [0.0838] | [0.0795] | [0.4482] | [0.2110] |
| | | | 38.5% | 36.5% | 22.6% | 35.8% |
| PS Item 7: | 1.40 | 1.75 (1.405) | 0.790 | 0.037 | 0.790 | 0.189 |
| Handwriting | (1.673) | | (0.2345, | (−0.4729, | (0.2896, | (−0.3151, |
| | | | 1.3449) | 0.5473) | 1.2897) | 0.6932) |
| | | | [0.0108] | [0.8721] | [0.0068] | [0.4115] |
| | | | 59.7% | 4.2% | 59.7% | 19.6% |
| Parameter | Mean (SD) | | LS Mean CFB (95% CI) [p-value] Mean CFB (SD)^{b} | | | |
| mADL^{c} | 8.60 | 12.17 | 9.789 | 1.350 | 7.589 | 4.353 |
| (Part B | (7.956) | (6.555) | (4.7981, | (−3.2686, | (1.0775, | (−1.8456, |
| Only) | | | 14.7796) | 5.9691) | 14.1001) | 10.5507) |
| | | | [0.0019] | [0.5192] | [0.0277] | [0.1450] |
| | | | 138.75 | 8.45 | 170.56 | 87.71 |
| | | | (74.985) | (34.208) | (159.685) | (126.281) |
| LS Mean Difference: Study drug - | | | −8.439 (−15.3856, −1.4916) | | −3.236 (−12.3264, 5.8538) | |
| Placebo (95% CI) | | | | | | |
| p-value: Treatment Group LS Mean | | | 0.0232 | | 0.4362 | |
| Difference | | | | | | |
| ADL | 14.80 | 22.17 | 10.455 | 0.877 | 9.255 | 3.267 |
| (Part B | (7.190) | (7.055) | (4.1039, | (−5.0097, | (1.8118, | (−3.9333, |
| Only) | | | 16.8060) | 6.7629) | 16.6981) | 10.4677) |
| | | | [0.0053] | [0.7401] | [0.0211] | [0.3262] |
| | | | 63.78 | 5.84 | 77.48 | 18.73 |
| | | | (60.046) | (16.276) | (66.631) | (24.838) |

| LS Mean Difference: Study drug - | −9.578 (−18.9020, −0.2546) | −5.988 (−16.8132, 4.8377) |
|---|---|---|
| Placebo (95% CI) | | |
| p-value: Treatment Group LS Mean | 0.0453 | 0.2387 |
| Difference | | |

ADL = Activities of Daily Living; CFB = change from baseline; CI = confidence interval; CTA = change in tremor amplitude (from randomization baseline); CULS = combined upper limb score; LS = least square; mADL = modified Activities of Daily Living; PS = performance subscale; PSTS = performance subscale total score; RWAS = randomized withdrawal analysis set; RWD = randomized withdrawal; TETRAS = The Essential Tremor Rating Assessment Scale; ULS = upper limb score.
^{a}The randomization baseline value was defined as the Day 42 post-dose value.
^{b}Change from randomization baseline.
^{c}mADL is referred to as TETRAS modified total score

TABLE 9
CGI-I Efficacy Endpoints: Baseline and Change from
Baseline Following Administration of Study Drug

| | CGI-I Score, n (%) | | | | | | | | |
|---|---|---|---|---|---|---|---|---|---|
| | | | 1 | | | | | | 7 |
| | | 0 | very | 2 | 3 | 4 | 5 | 6 | very |
| Assessment | | not | much | Much | minimally | no | minimally | much | much |
| Day | n | assessed | improved | improved | improved | change | worse | worse | worse |

Part A (FAS)^{a}

| | | | | | | | | | | |
|---|---|---|---|---|---|---|---|---|---|---|
| Day 7 | 6 | 0 | 0 | 1 | (16.7) | 1 (16.7) | 4 (66.7) | 0 | 0 | 0 |
| Day 14 | 6 | 0 | 0 | 1 | (16.7) | 5 (83.3) | 0 | 0 | 0 | 0 |

Part B OLT (FAS)^{a}

| | | | | | | | | | | | |
|---|---|---|---|---|---|---|---|---|---|---|---|
| Day 7 | 12 | 1 (8.3) | 0 | 1 | (8.3) | 5 (41.7) | 4 (33.3) | 1 | (8.3) | 0 | 0 |

| | | | | | | | | | | |
|---|---|---|---|---|---|---|---|---|---|---|
| Day 21 | 12 | 0 | 1 (8.3) | 1 | (8.3) | 5 (41.7) | 5 (41.7) | 0 | 0 | 0 |
| Day 42 | 11 | 0 | 0 | 4 | (36.4) | 4 (36.4) | 3 (27.3) | 0 | 0 | 0 |

Part B RWD (RWAS)^{b}

| Day 56 | 5 | 0 | 0 | 0 | 1 (20.0) | 3 (60.0) | 0 | | 1 (20.0) | 0 |
|---|---|---|---|---|---|---|---|---|---|---|
| (Placebo) | | | | | | | | | | |
| Day 56 | 6 | 0 | 0 | 0 | 1 (16.7) | 5 (83.3) | 0 | | 0 | 0 |
| (study drug) | | | | | | | | | | |
| Day 70 | 5 | 0 | 0 | 0 | 2 (40.0) | 3 (60.0) | 0 | | 0 | 0 |
| (Placebo) | | | | | | | | | | |
| Day 70 | 5 | 0 | 0 | 0 | 0 | 4 (80.0) | 1 | (20.0) | 0 | 0 |

(Study drug)

CGI-I = Clinical Global Impression-Improvement; FAS = full analysis set; OLT = open-label titration; PGI-C = Patient Global Impression of Change; RWAS = randomized withdrawal analysis set; RWD = randomized withdrawal.
^{a}Change from pre-dose baseline.
^{b}Change from randomization baseline.

TABLE 10
PGI-C Efficacy Endpoint: Baseline and Change from
Baseline Following Administration of Study Drug

| | PGI-C Score, n (%) | | | | | | | | |
|---|---|---|---|---|---|---|---|---|---|
| | | | 1 | | | | | | 7 |
| | | 0 | very | 2 | 3 | 4 | 5 | 6 | very |
| Study Part/ | | not | much | Much | minimally | no | minimally | much | much |
| Phase Day | n | assessed | improved | improved | improved | change | worse | worse | worse |

Part A (FAS)^{a}

| | | | | | | | | | | |
|---|---|---|---|---|---|---|---|---|---|---|
| Day 7 | 6 | — | 0 | 1 | (16.7) | 2 (33.3) | 2 (33.3) | 0 | 1 (16.7) | 0 |
| Day 14 | 6 | — | 0 | 1 | (16.7) | 5 (83.3) | 0 | 0 | 0 | 0 |

Part B OLT (FAS)^{a}

| | | | | | | | | | | |
|---|---|---|---|---|---|---|---|---|---|---|
| Day 7 | 13 | — | 0 | 1 | (7.7) | 5 (38.5) | 7 (53.8) | 0 | 0 | 0 |
| Day 21 | 12 | — | 0 | 1 | (8.3) | 5 (41.7) | 6 (50.0) | 0 | 0 | 0 |
| Day 42 | 11 | — | 0 | 3 | (27.3) | 4 (36.4) | 4 (36.4) | 0 | 0 | 0 |

Part B RWD (RWAS)^{b}

| Day 56 | 5 | | 0 | 0 | | 0 | 4 (80.0) | 0 | 0 | 1 (20.0) |
|---|---|---|---|---|---|---|---|---|---|---|
| (Placebo) | | — | | | | | | | | |
| Day 56 | 6 | | 0 | 0 | | 2 (33.3) | 3 (50.0) | 0 | 0 | 1 (16.7) |
| (Study drug) | | — | | | | | | | | |
| Day 70 | 5 | | 0 | 1 | (20.0) | 0 | 2 (40.0) | 2 (40.0) | 0 | 0 |

| | | | | | | | | | |
|---|---|---|---|---|---|---|---|---|---|
| (Placebo) | | — | | | | | | | |
| Day 70 | 5 | | 0 | 0 | 1 (20.0) | 3 (60.0) | 0 | 1 (20.0) | 0 |
| (Study drug) | | — | | | | | | | |

CGI-I = Clinical Global Impression-Improvement; FAS = full analysis set; OLT = open-label titration; PGI-C = Patient Global Impression of Change; RWAS = randomized withdrawal analysis set; RWD = randomized withdrawal.
^{a}Change from pre-dose baseline.
^{b}Change from randomization baseline.

TABLE 11
BAI and BDI-II Efficacy Endpoints: Baseline and Change
from Baseline Following Administration of Study Drug.

| | Assessment | | | | |
|---|---|---|---|---|---|
| Day, | Score, n (%) | | | | |
| Treatment | n | Minimal | Mild | Moderate | Severe |

BAI, n
Part B OLT (FAS)^{a}

| | | | | | | | | |
|---|---|---|---|---|---|---|---|---|
| Baseline | 14 | 6 | (42.9) | 6 | (42.9) | 2 | (14.3) | 0 |
| Day 7 | 13 | 7 | (53.8) | 3 | (23.1) | 3 | (23.1) | 0 |
| Day 21 | 12 | 9 | (75.0) | 1 | (8.3) | 2 | (16.7) | 0 |
| Day 42 | 11 | 9 | (81.8) | 1 | (9.1) | 1 | (9.1) | 0 |

Part B RWD (RWAS)^{b}

| Day 56, | 5 | 4 | (80.0) | 1 | (20.0) | 0 | 0 |
|---|---|---|---|---|---|---|---|
| Placebo | | | | | | | |
| Day 56, | 6 | 4 | (66.7) | 2 | (33.3) | 0 | 0 |
| Study drug | | | | | | | |
| Day 70, | 5 | 4 | (80.0) | 1 | (20.0) | 0 | 0 |
| Placebo | | | | | | | |
| Day 70, | 5 | 4 | (80.0) | 0 | | 0 | 1 (20.0) |
| Study drug | | | | | | | |

BDI-II Depressive Symptoms
Part B OLT (FAS)^{a}

| | | | | | | | |
|---|---|---|---|---|---|---|---|
| Baseline | 13 | 12 | (92.3) | 0 | 1 | (7.7) | 0 |

| | | | | | | | |
|---|---|---|---|---|---|---|---|
| Day 7 | 13 | 12 | (92.3) | 1 | (7.7) | 0 | 0 |
| Day 21 | 12 | 11 | (91.7) | 1 | (8.3) | 0 | 0 |

| | | | | | | |
|---|---|---|---|---|---|---|
| Day 42 | 10 | 10 | (100) | 0 | 0 | 0 |

Part B RWD (RWAS)^{b}

| Day 56, | 5 | 5 | (100) | 0 | | 0 | 0 |
|---|---|---|---|---|---|---|---|
| Placebo | | | | | | | |
| Day 56, | 6 | 6 | (100) | 0 | | 0 | 0 |
| Study drug | | | | | | | |
| Day 70, | 5 | 4 | (80.0) | 1 | (20.0) | 0 | 0 |
| Placebo | | | | | | | |
| Day 70, | 5 | 4 | (80.0) | 1 | (20.0) | 0 | 0 |
| Study drug | | | | | | | |

BAI = Beck Anxiety Inventory; BDI-II = Beck Depression Inventory-Second Edition; FAS = full analysis set; RWAS = randomized withdrawal analysis set; RWD = randomized withdrawal.
^{a}Change from pre-dose baseline.
^{b}Change from randomization baseline.

### Adverse Events

Treatment emergent adverse events (TEAEs) were mild to moderate and consistent with the safety profile for the program, as shown in the table below.

Overall, 85.7% of participants in Part A and 88.2% of participants in the open-label titration phase of Part B experienced at least 1 TEAE during the study, with 57.1% and 70.6% of participants, respectively, experiencing TEAEs considered related to study drug (see Table 12 below). A lower percentage of participants experienced TEAEs during the randomized withdrawal phase of Part B (20% in the placebo group and 16.7% in the study drug group).

Throughout the study, most TEAEs were mild or moderate in severity. One severe TEAE of increased ET post-randomization was experienced by a participant who had been randomized to the placebo group.

TEAEs leading to study drug withdrawal occurred in 6 participants in the open-label titration phase of Part B and 1 participant in Part A. All these AEs were mild or moderate in intensity, resolved, and 5 of these precipitating events occurred within the first 2 days of the 20 mg study drug treatment. One mild SAE of cyst was experienced by a participant in the open-label titration phase of Part B and was considered unrelated to study drug.

TABLE 12
Overall Summary of Treatment-Emergent Adverse Events (TEAEs)

| | Part A | Part | | | |
|---|---|---|---|---|---|
| | (overall) | B - OLT | | Part B - RWD | |
| | Study | Study | | | Study |
| Category, | drug | drug | | Placebo | drug |
| n (%) | N = 7 | N = 17 | | N = 5 | N = 6 |
| Any TEAE | 6 (85.7) | 15 | (88.2) | 1 (20.0) | 1 (16.7) |
| Mild | 6 (85.7) | 13 | (76.5) | 1 (20.0) | 1 (16.7) |
| Moderate | 1 (14.3) | 7 | (41.2) | 0 | 1 (16.7) |
| Severe | 0 | 0 | | 1 (20.0) | 0 |
| Any related to | 4 (57.1) | 12 | (70.6) | 1 (20.0) | 0 |
| study drug | | | | | |
| Any related to | 0 | 0 | | 0 | 0 |
| study procedure | | | | | |
| Any related to | 1 (14.3) | 1 | (5.9) | 1 (20.0) | 0 |
| disease under | | | | | |
| study | | | | | |
| Any serious | 0 | 1 | (5.9) | 0 | 0 |
| adverse events | | | | | |
| Any leading to | 1 (14.3) | 6 | (35.3) | 0 | 0 |

| | | | | |
|---|---|---|---|---|
| study drug | | | | |
| withdrawal | | | | |
| Any fatal SAEs | 0 | 0 | 0 | 0 |

N = sample size of group; n = number of individials occurring per adverse event category; OLT = open-label titration; RWD = randomized withdrawal; SAE = serious event; TEAE = treatment-emergent adverse event.

Overall, the most commonly experienced TEAEs in Part A were dizziness (4 participants, 57.1%) and headache (3 participants, 42.9%), with all but 1 TEAE of headache experienced by participants receiving 20 mg of the study drug.

TABLE 13
Summary of TEAEs by SOC and PT experienced by ≥2 participants
receiving study drug in any treatment group (Part A)

| | Study drug, | Study drug, | |
|---|---|---|---|
| SOC, n (%) | 20 mg | 40 mg | Overall |
| PT | N = 7 | N = 6 | N = 7 |
| Any TEAE | 6 (85.7) | 2 (33.3) | 6 (85.7) |
| Nervous System Disorders | 6 (85.7) | 1 (16.7) | 6 (85.6) |
| Dizziness | 4 (57.1) | 0 | 4 (57.1) |
| Headache | 3 (42.9) | 1 (16.7) | 3 (42.9) |
| Psychiatric Disorders | 2 (28.6) | 0 | 2 (28.6) |

Overall, the most commonly experienced TEAEs in the open-label titration phase of Part B were constipation (6 participants, 35.3%) and psychiatric and cognitive disorders (4 participants each, 23.5%) (see Table 14 below). The highest incidences of TEAEs in the open-label titration phase of Part B occurred at doses ≤60 mg of the study drug.

TABLE 14
Summary of Adverse Events by SOC and PT experienced by ≥2
participants receiving study drug in any treatment group (Part B)

| | Study drug dose | | | | | | | | | | |
|---|---|---|---|---|---|---|---|---|---|---|---|
| SOC, n (%) | 20 mg | | 40 mg | | 60 mg | | 80 mg | 100 mg | 120 mg | Overall | |
| PT | N = 17 | | N = 15 | | N = 12 | | N = 10 | N = 9 | N = 9 | N = 17 | |
| Any TEAE | 10 | (58.8) | 8 | (53.3) | 8 | (66.7) | 4 (40.0) | 1 (11.1) | 3 (33.3) | 15 | (88.2) |
| Nervous System Disorders | 8 | (47.1) | 4 | (26.7) | 8 | (66.7) | 2 (20.0) | 0 | 1 (11.1) | 12 | (70.6) |
| Dizziness | 4 | (23.5) | 1 | (6.7) | 2 | (16.7) | 0 | 0 | 0 | 6 | (35.3) |
| Cognitive disorder | 2 | (11.8) | 1 | (6.7) | 1 | (8.3) | 0 | 0 | 0 | 4 | (23.5) |
| Gastrointestinal Disorders | 3 | (17.6) | 3 | (20.0) | 0 | | 2 (20.0) | 1 (11.1) | 1 (11.1) | 8 | (47.1) |
| Constipation | 1 | (5.9) | 3 | (20.0) | 0 | | 1 (10.0) | 1 (11.1) | 1 (11.1) | 6 | (35.3) |
| General Disorders and | 2 | (11.8) | 2 | (13.3) | 3 | (25.0) | 0 | 0 | 0 | 5 | (29.4) |
| Administration Site Conditions | | | | | | | | | | | |
| Fatigue | 2 | (11.8) | 1 | (6.7) | 2 | (16.7) | 0 | 0 | 0 | 3 | (17.6) |
| Psychiatric Disorders | 1 | (5.9) | 2 | (13.3) | 1 | (8.3) | 0 | 0 | 1 (11.1) | 4 | (23.5) |

| | | | | | | | | | | |
|---|---|---|---|---|---|---|---|---|---|---|
| Insomnia | 1 | (5.9) | 2 | (13.3) | 0 | 0 | 0 | 0 | 3 | (17.6) |

| | | | | | | | | | | |
|---|---|---|---|---|---|---|---|---|---|---|
| Musculoskeletal and | 1 | (5.9) | 0 | 2 | (16.7) | 0 | 0 | 0 | 3 | (17.6) |

| | | | | | | | | | |
|---|---|---|---|---|---|---|---|---|---|
| Connective Tissue Disorders | | | | | | | | | |
| Vascular Disorders | 0 | 0 | 2 | (16.7) | 0 | 0 | 0 | 2 | (11.8) |

N = sample size of group; OLT = open-label titration; PT = preferred term; SAE = serious adverse event; SOC = system organ class; TEAE = treatment-emergent adverse event.
SOCs are ordered in decreasing frequency in the Part B - OLT group, with PTs listed in decreasing order within each SOC.

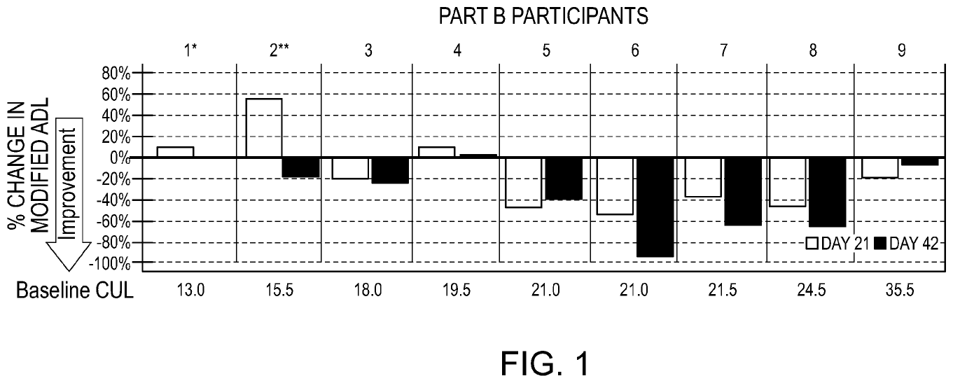

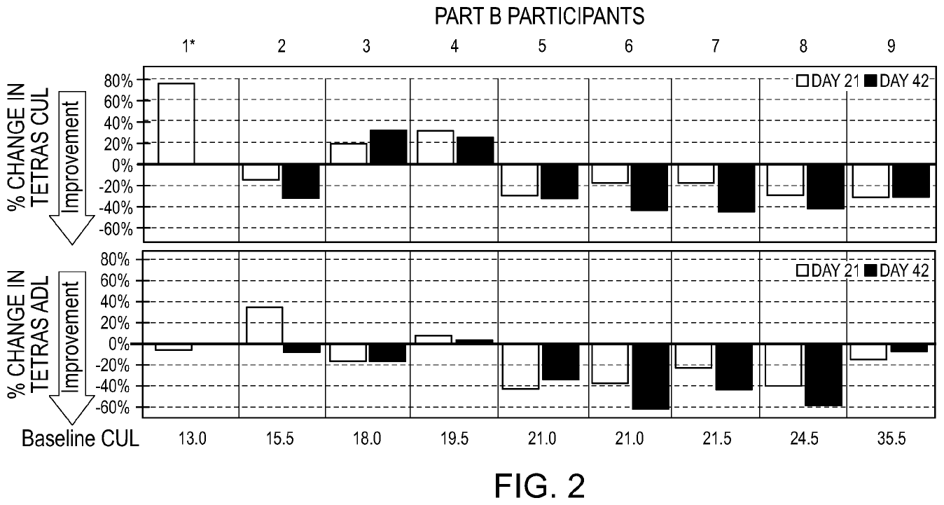

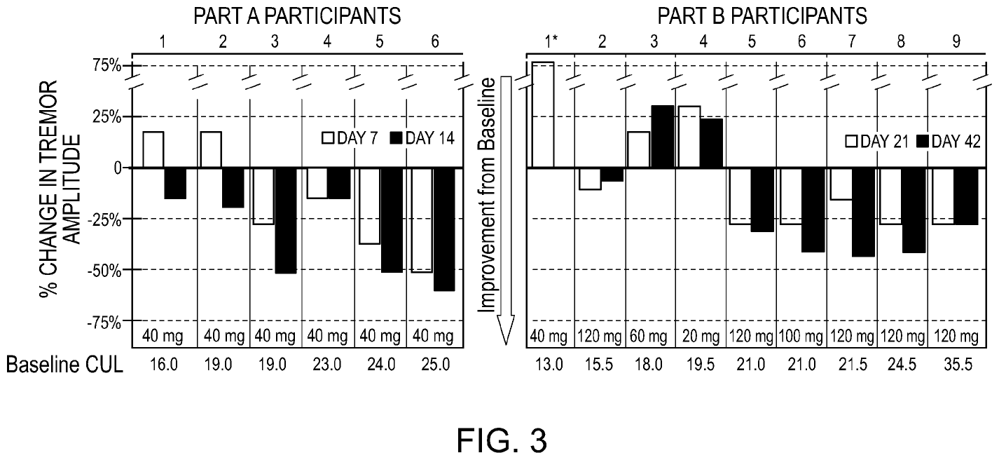

Preliminary Part B data showing modified activities of daily living (ADL) scores as compared to baseline combined upper limb (CUL) score is provided in FIG. 1. TETRA CUL and TETRAS ADL are provided in FIG. 2. TETRAS CUL is provided in FIG. 3.

### DISCUSSION AND CONCLUSIONS

This trial was conducted in 2 parts, enrolling adult male and female participants who had a clinical diagnosis of ET for at least 3 years at screening, with an age of onset that was earlier than 65 years of age. Part A was open-label and assessed the safety and tolerability of the study drug, as well as the overall magnitude and pattern of change in ET severity. Daily dose levels were titrated from 20 mg to 40 mg. Part B consisted of both an open-label titration phase and a randomized, double-blind, placebo-controlled withdrawal phase. Part B assessed the safety and tolerability of the study drug, as well as the overall magnitude and pattern of change in ET severity and the duration of that effect. Daily dose levels were titrated from 20 mg to up to 120 mg during the open-label phase. In the randomized, double-blind, placebo-controlled withdrawal phase, participants were either maintained on their final open-label dose or switched to placebo.

### Efficacy

The primary objective of this study was to evaluate the efficacy of the study drug on upper limb tremor in participants with ET.

### Part A Open-Label Study

In Part A, dosing with the study drug led to marked reduction (i.e., improvement) in the primary efficacy parameter, TETRAS upper limb score on Day 7 (after 7 days of 20 mg QAM dosing) that was further reduced on Day 14 (after an additional 7 days of 40 mg QAM dosing). These improvements were observed alongside reductions in upper limb tremor amplitude of 23.9% and 42.8%, respectively, in upper limb tremor amplitude.

Similar improvements were observed for combined upper limb and TETRAS performance subscale total scores. Improvements were most pronounced for all TETRAS performance subscale-derived measures (including upper limb and combined upper limb scores) in participants who had upper limb scores ≥10 at baseline.

As expected, all TETRAS performance subscale-derived scores returned to near pre-dose baseline levels after a 1-week washout period, on Day 21. This finding supports the need for continued the study drug dosing to achieve efficacy.

### Part B Open-Label Titration Phase

In the open-label titration phase of Part B, a reduction (ie, improvement) in TETRAS upper limb score was observed after only 7 days of 20 mg dosing of the study drug. Mean improvement became increasingly pronounced over time, through Day 42, as participants titrated to their highest tolerated dose (ranging from 20 to 120 mg of the study drug). As in Part A, similar improvements were observed for all TETRAS performance subscale-derived measures (including upper limb and combined upper limb scores), with improvements most pronounced in participants who had upper limb scores ≥10 at baseline.

TETRAS ADL and mADL scores were also reduced as early as Day 7, with continued improvement observed through Day 42. These findings, combined with improvements in quality of life measures such as QUEST, CGI-I, CGI-S, and PGI-C support functional improvement in ET symptom severity during the titration phase (at Day 7 and Day 21) that continued to improve after participants had received 2 weeks of stable dosing at either 120 mg of the study drug or the highest tolerated dose on Day 42. Comparable improvements in ADL, mADL, and quality of life measures were observed in participants who had upper limb scores ≥10 at baseline, reflecting functional improvement in participants over a range of ET severity levels at baseline.

### Part B Randomized-Withdrawal Phase

In the Part B randomized withdrawal phase, comparison to the randomization baseline on Day 42 revealed an increase (i.e., worsening) in TETRAS combined upper limb and performance subscale total scores on Day 56 that were larger in the placebo group compared with the study drug group indicating a maintenance of effect in the continuously treated participants. The upper limb score change from randomization baseline on Day 42 to Day 56 was comparable between the placebo and the study drug groups.

Comparison of mADL scores to the randomization baseline on Day 42 revealed an increase (i.e., worsening) in the mADL total score on Day 56 that was greater in the placebo group compared with the study drug group, with an LS mean difference between treatment groups (the study drug—placebo) of −8.439 and a 95% CI below zero (−15.3856, −1.4916) demonstrating a nonzero change from baseline. A similar trend was observed for ADL scores over the same timeframe. Scores for QUEST, CGI-I, and CGI-S, also support sustained efficacy in the study drug group, with deterioration relative to randomization baseline for those randomized to the placebo group.

All TETRAS performance subscale-derived, TETRAS ADL-derived, and quality of life measures returned to near pre-dose baseline levels after a 1-week washout period, on Day 70, supporting the need for continued study drug dosing to achieve sustained efficacy.

### Quality of Life, Symptom Severity, and Anxiety and Depression

Quality of life measured by QUEST and symptom severity measured by CGI-I and PGI-C improved for participants receiving study drug from pre-dose baseline to Day 56 in Part B. There was a shift in BAI scores from mild to minimal anxiety (i.e., improvement) in the Part B open-label titration phase. The majority of participants in both treatment groups had BDI-II scores reflecting minimal depressive symptoms from pre-dose baseline to Day 70, with no clinically meaningful change over time.

### Pharmacokinetics

Measurable study drug plasma concentrations were obtained for all participants 1-hour post-dose. On Day 42 steady-state exposure was achieved, with similar study drug plasma concentrations on Day 56 (379.4 ng/mL versus 344.1 ng/mL), consistent with the characterized PK profile of the study drug. After 56 days of daily dosing, a 14-day washout period resulted in undetectable study drug plasma concentrations.

### Safety

The safety profile of the study drug was similar between Part A and the open-label titration phase of Part B. In these study parts most participants experienced at least 1 TEAE and all were mild to moderate in intensity. One mild SAE of cyst was experienced by a participant in the open-label titration phase of Part B and was considered unrelated to study drug. While 7 participants experienced AEs leading to study drug withdrawal, 2 of the AEs were considered unrelated (anxiety and cyst), 4 of the AEs occurred during the first 2 days of dosing at the 20 mg dose of the study drug, and 3 of the participants experiencing these AEs were from a site that was ultimately suspended for repeatedly enrolling ineligible subjects.

In the randomized withdrawal phase of Part B, only 1 participant each in the study drug and placebo groups experienced a TEAE. Thus, as expected, TEAEs are more infrequent in participants who have titrated up to their maximally tolerated dose. Given that all AEs leading to discontinuation occurred during titration, lower dose levels (5 mg and 10 mg) will be administered in future studies at 1-week dosing intervals to initiate treatment with the study drug, before escalating to each participant's highest tolerated dose. Focusing on early tolerability so that participants can reach a stable, effective dose will continue to be an emphasis of the clinical development program.

The most common TEAEs in Part A were dizziness and headache, with all events except for one event of headache occurring at the lower 20 mg dose of the study drug. The most common TEAEs in the open-label titration phase of Part B were constipation, cognitive disorders, and psychiatric disorders, with the highest overall incidence of TEAEs occurring at doses ≤60 mg of the study drug. The observed AE of constipation in Part B may suggest that sustained dosing with a T-type calcium channel inhibitor, such as the study drug, may impact colon motility, as described for nifedipine and verapamil, but will require further investigation. No TEAEs had a date of onset during the randomized withdrawal phase of Part B in more than 1 participant. However, all participants receiving the study drug in the randomized-withdrawal phase of Part B continued to experience some ongoing AEs that had emerged during the open-label titration period, most commonly constipation in 3 participants and photosensitivity/photophobia in 2 participants.

Drug-related TEAEs experienced by ≥2 participants included dizziness (3 participants in Part A and 6 participants in the open-label titration phase of Part B), cognitive disorder (4 participants in the open-label titration phase of Part B), and paraesthesia (2 participants in the open-label titration phase of Part B). All these events were mild or moderate in severity.

### CONCLUSIONS

In this study, the study drug markedly reduced upper limb tremor in participants with ET at Day 7, with continued improvement at Day 14, as assessed by TETRAS upper limb score. These findings were supported by similar improvements in TETRAS performance subscale, TETRAS ADL, mADL, and measures throughout Part A and the open-label titration phase of Part B.

Overall, the study drug was generally safe and well tolerated at doses up to 120 mg. The accelerated titration regimen used in this study resulted in discontinuation of a third of participants during the titration period. Therefore, utilization of a more gradual titration regimen will be used to enhance early tolerability of the study drug in future studies.

### Example 2: A Phase 2, Randomized, Double-Blind, Placebo-Controlled, Dose Range Finding Clinical Trial to Evaluate the Tolerability, Safety, and Efficacy of a Compound of Formula (I) in the Treatment of Adults with Essential Tremor

This multi-center, randomized, double-blind, placebo-controlled, dose-range-finding clinical trial was conducted to assess the efficacy, safety, and tolerability of the HCl salt compound of Formula (I) in participants aged 18 years or older who have a diagnosis of essential tremor (ET) and have had symptoms for at least 3 years. Participants were randomized to receive either 56 days of the HCl salt compound of Formula (I) or a matching placebo.

The clinical trial consisted of 3 study periods: Screening/Baseline, Intervention, and Safety Follow-up.

### Screening/Baseline Period

The Screening Period was up to 28 days in duration (Day −28 to Day −1) but could be extended by 14 additional days (Day −42 to Day −1) for those participants needing to discontinue primidone. To be eligible, participants who were taking primidone at Screening had to successfully discontinue this drug a minimum of 14 days prior to the first dose of study drug.

Key screening assessments included medical history, demographics, prior and concomitant medications, physical examination, drug/alcohol screen, clinical laboratory evaluations, 12-lead ECG, vital signs, C-SSRS, and assessments of ET severity using TETRAS PS (including a video recording for independent review of eligibility), TETRAS ADL, and CGI-S.

### Intervention Period

Participants who continued to meet all clinical trial entry criteria on Day 1 were randomized to receive double-blind treatment with the HCl salt compound of Formula (I) or placebo every morning (QAM) from Day 1 through Day 56. To allow for dose titration in 2 of the treatment regimens in the HCl salt compound of Formula (I) group (120 mg and 60 mg), participants were randomized to 1 of 3 fixed-dose regimens or placebo in a 1:1:1:1 ratio.

Throughout the Intervention Period (Day 1 to Day 56), participants who chose to stop dosing or were required to stop dosing entered the Follow-up Period if they did not withdraw their consent to participate.

On Day 1, participants received 6 tablets of study drug QAM. TETRAS PS, TETRAS ADL, The Essential Tremor Performance Based Test (ET Performance Based Test), CGI-S, QUEST, BDI-II, BAI, and Mobile Phone-Based Video Tremor Tasks were performed before dosing. Additional assessments were performed pre-dose including pharmacokinetic (PK) sampling.

Participants continued taking 6 tablets of study drug QAM throughout the trial. On Days 14, 28, 42, and 56, participants returned to the clinic for safety and efficacy assessments. On Days 14, 28, 42, and 56, participants took study drug QAM in the clinic after pre-dose PK samples, and any pre-dose assessments were collected. Key efficacy assessments at these visits included a selection of the following endpoints: TETRAS PS or CUL assessment, TETRAS ADL, QUEST, ET Performance Based Test, BAI, BDI-II, CGI-S, CGI-I, and PGI-C. On Days 7, 43, 44, 54, and 55, participants performed an Archimedes spiral drawing and a handwriting sample at home. On Days 43, 44, 54, and 55, participants performed Mobile Phone-Based Video Tremor Tasks. On Days 7, 21, 35, and 54 participants received a telephone call from site staff to ask about AEs and changes in concomitant medications.

### Safety Follow-Up Period

The Safety Follow-up Period was from Day 57 to Day 70. At the end of the Safety Follow-up Period, participants returned to the study site on Day 70 (±1 day) for the final clinical trial assessments.

### Number of Participants

Approximately 112 participants were randomized (approximately 28 to each of the 4 groups) to achieve approximately 88 evaluable participants (i.e., 22 per treatment group).

### Duration of Clinical Trial

TABLE 15

| | |
|---|---|
| Screening period | Up to 28 days (up to +14 days washout |
| | if discontining primidone) |
| Intervention period | 56 days |
| Safety Follow-up period | 14 days |
| Total participation | Up to 98 days (up to 112 days |
| | if discontinuing primidone) |

### Inclusion Criteria

Among the list of inclusion criteria, to be eligible to participate in this clinical trial a participant at Screening had a clinical diagnosis of ET including:
a. tremor syndrome of bilateral upper limb action tremor,
b. at least 3 years in duration
c. with or without tremor in other locations (e.g., head, voice, or lower limbs),
d. If the symptoms and signs are judged by the investigator to be due to the diagnosis of ET, it is acceptable for them to also have one or more of the following ET plus signs:
   i. mild dystonic posturing,
   ii. mild rest tremor in the setting of advanced ET and in the absence of other features of Parkinsonism,
   iii. intention tremor,
   iv. mild increase in tandem gait difficulty.

Eligible participants at Screening also had a TETRAS upper limb score (i.e., sum of bilateral upper limb items 4a, 4b, and 4c) of ≥10 as rated by the Investigator at Screening and Baseline.

If currently receiving any medication for ET, eligible participants were on a stable dose of any of these medications for ET for 1 month prior to Screening and were willing to maintain stable doses throughout the trial. If receiving primidone for ET, eligible participants were willing and able to discontinue 14 days prior to Day 1.

### Test Product, Reference Therapy, Administration

The HCl salt compound of Formula (I) was supplied as 20 mg modified-release tablets. Matching placebo was also supplied. Study drug was administered orally and provided in pre-packaged containers to the participants. All participants received 6 tablets per day. Participants in one of the study drug groups received a combination of the HCl salt compound of Formula (I) and matching placebo tablets, with the number of each tablet type depending on the assigned dose regimen and study day. Participants in the placebo group received 6 tablets of matching placebo on all dosing days.

### Dose/Route/Regimen

Eligible participants were randomized to receive 1 of 3 study drug dosing regimens (20 mg, 60 mg, or 120 mg) or placebo, administered orally QAM. This randomized, double-blind, placebo-controlled, dose range finding clinical trial assigned participants to receive 56 days of treatment with either study drug or placebo every morning. To achieve dose levels above 20 mg (i.e., 60 mg and 120 mg), fixed titration regimens were used. Participants were not allowed to adjust the number of tablets per day. See Table 16 below for the dosing regimens.

TABLE 16
Dosing Sequence by Regimen

| | Dosing Sequence | | | | | |
|---|---|---|---|---|---|---|
| | Days on | Days on | Days on | Days on | Days on | Days on |
| | 20 mg | 40 mg | 60 mg | 80 mg | 100 mg | 120 ing |
| Regimen | Dose | Dose | Dose | Dose | Dose | Dose |
| Regimen 1 (active drug) | 3 | 4 | 7 | 7 | 7 | 28 |
| Regimen 2 (active drug) | 3 | 4 | 49 | — | — | — |
| Regimen 3 (active drug) | 56 | — | — | — | — | — |

| | |
|---|---|
| Placebo | Dosed with placebo every day during Intervention Period (56 days) |

### Objectives and Endpoints

The objectives and endpoints are summarized in the following table.

TABLE 17
Objectives and Endpoints

| | |
|---|---|
| Objective | Endpoint |

Primary

| | |
|---|---|
| To assess for the presence of a tolerability | Incidence and severity of AEs including |
| dose-response relationship for study drug in | discontinuations of study drug due to AEs |
| participants with ET | |

Secondary

| | |
|---|---|
| To assess for the presence of an efficacy | Change from baseline to Day 56 on the |
| dose-response relationship for study drug in | Essential Tremor Rating Assessment Scale |
| participants with ET | (TETRAS) combined upper limb (CUL) score |
| | comprised of upper limb, spiral drawing, |
| | handwriting, and dot approximation tasks as |
| | scored by site rater |
| | Change from baseline to Day 56 on the |
| | TETRAS ADL score |

Other
Efficacy

| | |
|---|---|
| To evaluate the efficacy of study drug | Change from baseline to Day 56 and all other |
| compared to placebo in participants with ET | post dose timepoints in the: o TETRAS CUL |
| | score (all other post dose timepoints other than |
| | Day 56) |
| | TETRAS ADL subscale score (all other post |
| | dose timepoints other than Day 56) |
| | TETRAS Performance subscale (PS) total |
| | score and individual items scores |
| | Modified combined TETRAS ADL and |
| | TETRAS PS spirography and handwriting |
| | score Quality of Life in Essential Tremor |
| | Questionnaire (QUEST) total and subscale |
| | scores |
| | The Essential Tremor Performance Based Test |
| To evaluate the efficacy of study drug | Change from baseline to Day 56 and all other |
| compared to placebo on global measures of | post dose timepoints in Clinical Global |
| disease state in participants with ET | Impression-Severity (CGI-S) score |
| | Patient Global Impression of Change (PGI-C) |
| | and Clinical Global Impression-Improvement |
| | (CGI-I) scores at each post-baseline |
| | assessment |
| To evaluate the efficacy of study drug | Change from baseline to Day 56 and all other |
| compared to placebo on measures of mood and | post dose timepoints in the: |
| anxiety in participants with ET | Beck Depression Inventory-Second Edition |
| | (BDI-II) total and subscale scores |
| | Beck Anxiety Inventory (BAI) total score |
| To evaluate the efficacy of study drug | Change from baseline to each post-baseline |
| compared with placebo on remote assessments | assessment in TETRAS PS spiral drawing and |
| of upper limb tremor in participants with ET | handwriting item scores as performed at home |
| | and scored by site rater |
| To evaluate the efficacy of study drug | Change from baseline in each post-baseline |
| compared with placebo on mobile phone | assessment in computer-based video analytics |
| captured videos in participants with ET | |

Safety

| | |
|---|---|
| To evaluate the safety of study drug compared | Changes in vital sign measurements |
| to placebo | Changes in clinical laboratory results |
| | Changes in electrocardiogram (ECG) |
| | parameters |
| | Incidence of Columbia-Suicide Severity Rating |
| | Scale (C-SSRS) measured suicidal ideation or |
| | behavior |

PK

| | |
|---|---|
| To determine plasma concentrations of study | Plasma study drug and metabolite |
| drug and its metabolites in participants with ET | concentrations over time |

### Example 3. Effect of the HCl Salt of Compound of Formula (I) Alone or in Combination with Propranolol on Tremor Activity in Rats

This study sought to evaluate the effects of the HCl salt of the compound of Formula (I) (the “study drug”) administered alone (1 mg/kg, p.o.) or in combination with propranolol (1 and 3 mg/kg, i.p. at 20 min prior) in Harmaline (10 or 30 mg/kg, i.p.) induced tremor activity measured with a piezoelectric plate in male Sprague Dawley (SD) rats.

Male SD rats had a body weight of about 250 g at time of testing. Rats were fed ad lib and kept at a 12:12 light-dark cycle throughout the study. There were N=15 in each group. Harmaline and propranolol were each formulated with saline using a volume of 5 ml/kg bodyweight. Vehicle for the study drug (1 mg/kg) was 0.5% methyl cellulose/0.1% Tween-80 in water. See tables below.

Table 18 provides the doses for the first study based on the protocols set forth in this example. Table 19 provides the adjusted doses for a repeated (second) study based on the protocols set forth in this example.

TABLE 18

| Group | T1 = −60 min p.o. | T2 = −20 min i.p. | T3 = 0 min i.p. |
|---|---|---|---|
| 1 | Vehicle | Saline | Saline |
| 2 | Vehicle | Saline | Harmaline |
| | | | (30 mg/kg) |
| 3 | Study Compound | Saline | Harmaline |
| | (1 mg/kg) | | (30 mg/kg) |
| 4 | Vehicle | Propranolol | Harmaline |
| | | (1 mg/kg) | (30 mg/kg) |
| 5 | Vehicle | Propranolol | Harmaline |
| | | (3 mg/kg) | (30 mg/kg) |
| 6 | Study Compound | Propranolol | Harmaline |
| | (1 mg/kg) | (1 mg/kg) | (30 mg/kg) |
| 7 | Study Compound | Propranolol | Harmaline |
| | (1 mg/kg) | (3 mg/kg) | (30 mg/kg) |

TABLE 19

| Group | T1 = −60 min p.o. | T2 = −20 min i.p. | T3 = 0 min i.p. |
|---|---|---|---|
| 1 | Vehicle | Saline | Saline |
| 2 | Vehicle | Saline | Harmaline |
| | | | (30 mg/kg) |
| 3 | Study Compound | Saline | Harmaline |
| | (0.3 mg/kg) | | (30 mg/kg) |
| 4 | Vehicle | Propranolol | Harmaline |
| | | (0.1 mg/kg) | (30 mg/kg) |
| 5 | Vehicle | Propranolol | Harmaline |
| | | (0.3 mg/kg) | (30 mg/kg) |
| 6 | Study Compound | Propranolol | Harmaline |
| | (0.3 mg/kg) | (0.1 mg/kg) | (30 mg/kg) |
| 7 | Study Compound | Propranolol | Harmaline |
| | (0.3 mg/kg) | (0.3 mg/kg) | (30 mg/kg) |

### Methods

1) Rats were allowed to acclimate in standard laboratory animal facility conditions for at least 5-7 days prior to use.
2) Rat was put in a Plexiglas chamber with a piezoelectric plate attached at the bottom to transduce tremor behavior into electronic signal. Electric signal of tremor is 100× amplified with A-M Systems (model 1700) and digitized with CED-micro 1401 at a sampling rate of 512 Hz and saved in “smr” format for offline analyses using Spike-2 software (version 7.07).
3) One day prior to the first testing day, a naive rat was put in the testing chamber to confirm that the spontaneous activity baseline was within the range of 3000-10000 uv, as this sensitivity level generally allows all the tremor signal to be detected with the system.
4) There were 7 groups in the experiment (7×15=105 rats), done on 7 days with balanced group arrangement each day.
5) The animals were randomly assigned into each treatment group and across the testing days.
6) On each testing day, each rat was allowed at least a 30-minute acclimation session in the test room.
7) Prior to testing, the rat was placed into the testing chamber for 5 minutes with the system offset to ensure the baseline activity is within the accepted range (3000-10000 uv).
8) Rat was administered P.O. with study compounds and vehicle (60 min prior), and then administered i.p. with propranolol at 20 minutes prior to harmaline injection.
9) After that, the rat was kept in the test chamber to permit a 20-minute baseline recording (−20 to 0 min) before harmaline (30 mg/kg) treatment (pre-harmaline). 8 rats were tested simultaneously by using 8 testing chambers in a counterbalanced order.
10) After the 20-minute baseline recording (−20 to 0 min), each rat was i.p. dosed with harmaline (30 mg/kg).
11) Post-harmaline effect was measured for 20 min (10-30 min).
12) Immediately after completion of tremor recording, the rat was transferred to a designated necropsy room which is close to the procedure room. The rats were then anesthetized with CO₂ and blood was collected via the left ventricle (˜90 min post-harmaline).
13) The animals were sacrificed, and relevant tissues were removed for immediate dissection and kept in the dry ice.

### Piezoelectric Plate Signal Analysis

1) Power spectral density analysis:
Electrical tremor signal was processed with fast Fourier transform (FFT) at resolution of 0.5 Hz bin.
PZ power density was computed from 20 minutes before and 20 minutes after harmaline injection (10-30 minutes) in the frequency range between 1 to 40 Hz.
Non-normalized power density after harmaline administration as plotted in 0.5 Hz bins.
Changes of power density after harmaline administration was normalized to 1) the average of the 10-minute baseline; and 2) the average of the 20 minute baseline of each rat in 0.5 Hz bins.
Average power density over the 8-13 Hz band and in 9-12 Hz band was expressed in percent change relative to baseline value individually and plotted as a bar graph.
Average maximum power density across the 8-13 Hz band in 0.5 Hz bins was calculated and plotted as a bar graph.
   2) Power-time analysis:
Electrical tremor signal was processed with fast Fourier transform (FFT) in time bin of 1 minute through the frequency range 8-13 Hz and 9-12 Hz.
Non-normalized average power density over the 8-13 Hz band and in the 9-12 Hz band from 20 minutes pre-harmaline to 20 minutes post-harmaline administration in 1-minute bins was plotted as a line graph.
Raw data of non-normalized power density over the 8-13 Hz band and in the 9-12 Hz band from 20 minutes pre-harmaline to 20 minutes post-harmaline administration from individual animals in each group was collected.
Changes of power density after harmaline administration were normalized to 1) the average of the 10-minute baseline; and 2) the average of the 20 minute baseline of each rat in a time bin of 1 min.
Area under the power change-time curve (AUC) over the 20-minute time period after harmaline administration was calculated for each rat and the average of AUC was plotted as a bar graph.
Dominant EEG frequency over time period from pre-harmaline to 20 minutes post-harmaline was collected.

Plasma and brain tissue sample collection (at 90 min post dosing of study compound) was evaluated in 15 test rats. See table below.

TABLE 20

| | T0 min: HCl | | |
|---|---|---|---|
| | salt of | | |
| | Compound of | T40 min: | T90 min: |
| Group | Formula (I) | Propranolol | Satellite PK |
| 3 (n = 15) | 1 mg/kg | saline | Analyze |
| | | | for study |
| | | | compound |
| 4 (n = 15) | Vehicle | 1 mg/kg | Analyze for |
| | | | Propranolol |
| 5 (n = 15) | Vehicle | 3 mg/kg | Analyze for |
| | | | Propranolol |
| 6 (n = 15) | 1 mg/kg | 1 mg/kg | Analyze |
| | | | for both |
| 7 (n = 15) | 1 mg/kg | 3 mg/kg | Analyze |
| | | | for both |

N=15 in each group. Plasma and brain tissue samples were collected immediately after harmaline testing. Plasma sample number: 5×15=75. Brain tissue sample number: 5×15=75.

### Results

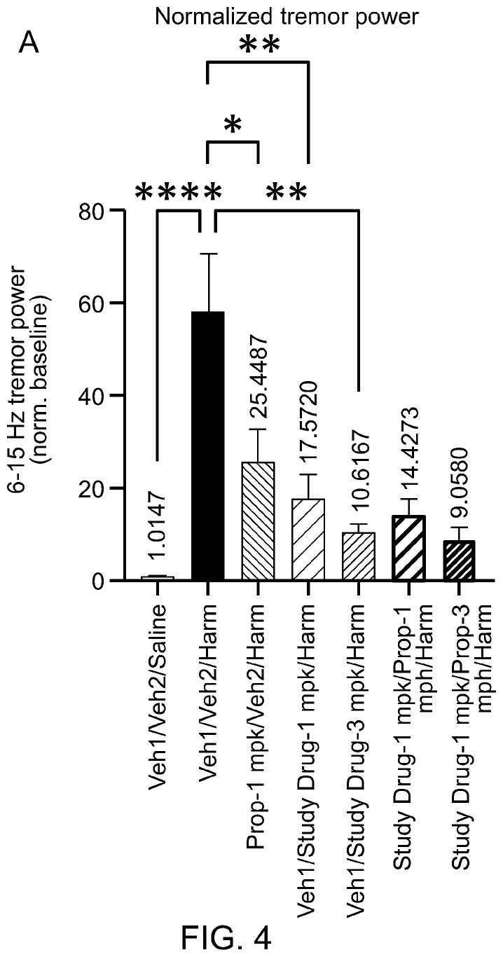

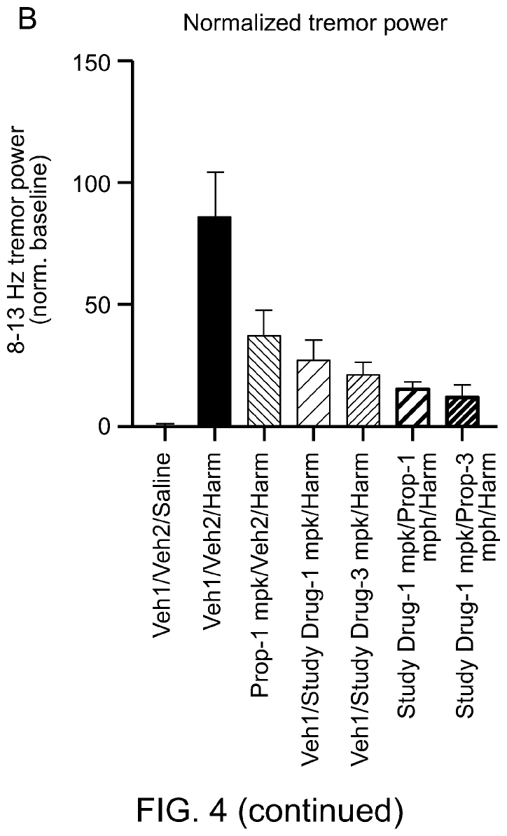

The results of the study are presented in FIGS. 4-6. Specifically, FIG. 4, Panel A is a bar graph showing tremor power measured in the 8-13 Hz band in harmaline-treated rats that were administered compound of 1 mg/kg of the compound of Formula (I) alone, or in combination with 1 mg/kg or 3 mg/kg propranolol. FIG. 4, Panel B is a bar graph showing tremor power measured in the 6-15 Hz band in harmaline-treated rats that were administered compound of 1 mg/kg of the compound of Formula (I) alone, or in combination with 1 mg/kg or 3 mg/kg propranolol. The results presented in FIG. 4, Panels A and B demonstrate that the compound of Formula (I) significantly reduces harmaline-induced tremor when administered alone or with concomitant administration of propranolol. Further, plasma and brain concentrations of the compound of Formula (I) and propranolol were consistent with those measured in previous studies.

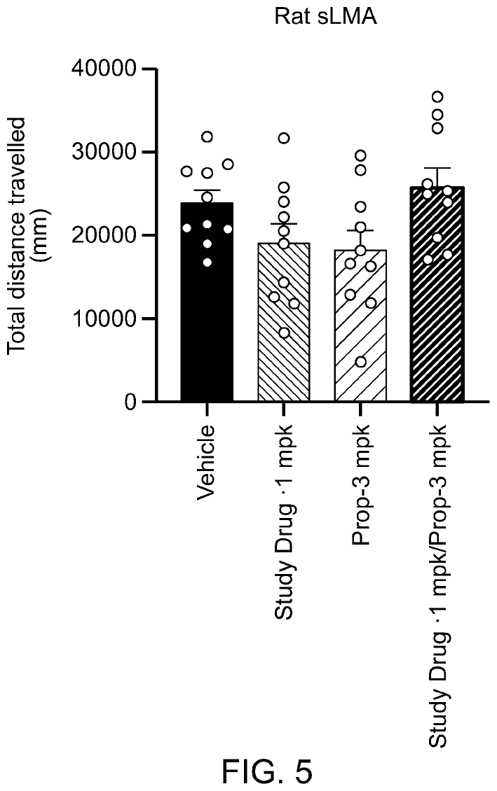

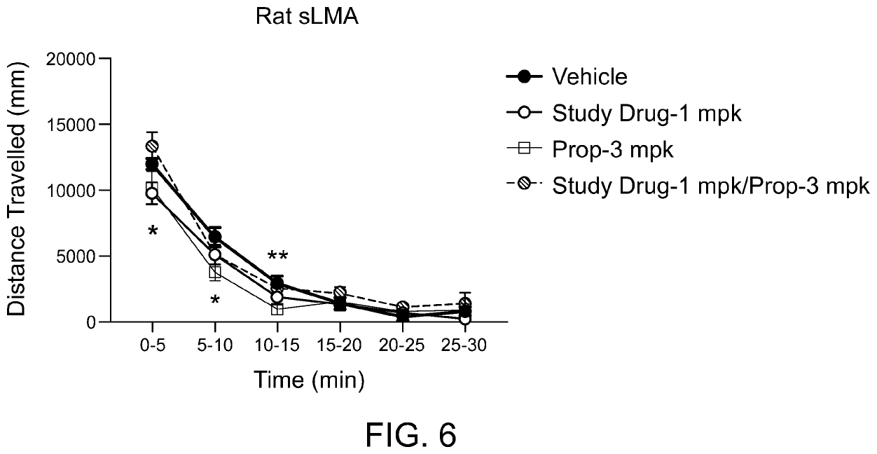

FIG. 5 is a bar graph showing sLMA as total distance travelled (in mm) measured in rats treated with the compound of Formula (I) and propranolol alone or in combination. FIG. 6 is a graph showing sLMA as total distance travelled (in mm) over time measured in rats treated with the compound of Formula (I) and propranolol alone or in combination. The results presented in FIGS. 5 and 6 indicate that the compound of Formula (I) or propranolol alone or in combination do not significantly reduce total sLMA in rats. Plasma concentration and brain tissue concentration of the compound of Formula (I) and propranolol were consistent with those measured in previous studies.

### Example 4. Effect of Formula (I) and Propranolol in Rat sLMA Assay

The goal of this study was to determine the effect of the compound of Formula (I) and propranolol alone and in combination on rat spontaneous locomotor activity (sLMA). For both propranolol and the compound of Formula (I), 1 mg/kg of the compound of Formula (I) and 3 mg/kg propranolol were tested. Rat sLMA was measured from 0-30 min (0 minutes is 60 minutes after administration of the compound of Formula (I) and 20 minutes after administration of propranolol).

TABLE 21

| Group | T1 = −60 min p.o. | T2 = −20 min i.p. |
|---|---|---|
| 1 | Vehicle | Saline |
| 4 | Vehicle | Propranolol |
| | | (1 mg/kg) |
| 5 | Vehicle | Propranolol |
| | | (3 mg/kg) |
| 7 | Formula (I) | Propranolol |
| | (1 mg/kg) | (3 mg/kg) |

FIG. 5 is a bar graph showing sLMA as total distance travelled (in mm) measured in rats treated with the compound of Formula (I) and propranolol alone or in combination. FIG. 6 is a graph showing sLMA as total distance travelled (in mm) over time measured in rats treated with the compound of Formula (I) and propranolol alone or in combination. The results presented in FIGS. 5 and 6 indicate that the compound of Formula (I) or propranolol alone or in combination do not significantly reduce total sLMA in rats. Plasma concentration and brain tissue concentration of the compound of Formula (I) and propranolol were consistent with those measured in previous studies.

### Example 5. Effect of Compound of Formula (I) (1, 3 and 10 mg/kg, p.o.) or Propranolol on Multiple Endpoints with Harmaline in SD Rats: Tremor Assessment, Open Field, Rotarod, and Wire Grip Tests

Each of six groups of male SD rats were subjected to a tremor assessment, followed by three ADL-like measures including open field test, rotarod test, and wire grip test after receiving doses of either vehicle, Formula (I), or propranolol, followed by either saline or harmaline, as outlined below in Table 22 below.

TABLE 22

| Group | T1 = −40 min | T2 = −20 min | T3 = 0 min |
|---|---|---|---|
| 1 | Vehicle | — | Saline |
| 2 | Vehicle | — | Harmaline |
| | | | (30 mg/kg) |
| 3 | Formula (I) | — | Harmaline |
| | (1 mg/kg) | | (30 mg/kg) |
| 4 | Formula (I) | — | Harmaline |
| | (3 mg/kg) | | (30 mg/kg) |
| 5 | Formula (I) | — | Harmaline |
| | (10 mg/kg) | | (30 mg/kg) |
| 6 | — | Propranolol | Harmaline |
| | | (10 mg/kg) | (30 mg/kg) |

Twenty minutes after administration of harmaline or saline, the rats were subjected to a tremor assessment using the score criteria outlined in the table below.

TABLE 23

| Score | Tremor | Description |
|---|---|---|
| 0 | No tremor | N/A |
| 1 | Occasional tremor | affecting only the head and neck |
| 2 | Intermittent | occasional tremor affecting all body parts |
| 3 | Persistent | tremor affecting all body parts |
| 4 | Severe | persistent tremor rendering the animal |
| | | unable to stand and/or walk |

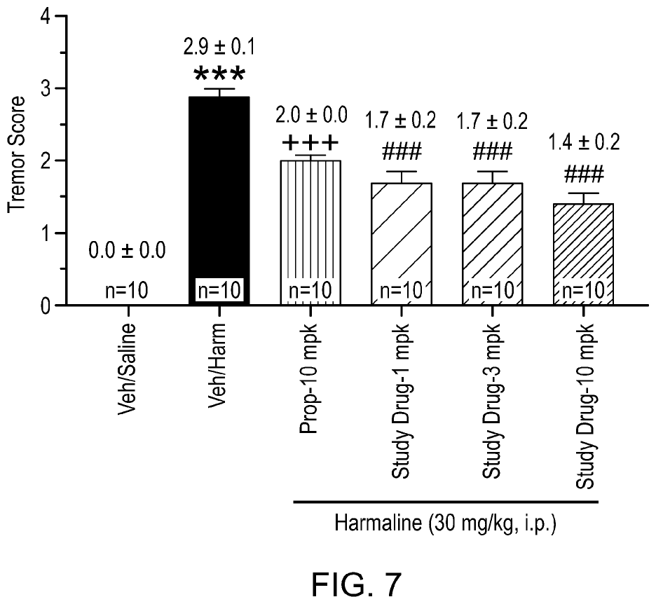

The results of the experiment are shown in FIG. 7. Specifically, FIG. 7 is a bar graph showing the tremor score measured in rats that were administered 30 mg/kg harmaline and also administered 10 mg/kg propranolol or 1 mg/kg, 3 mg/kg or 10 mg/kg study drug. As shown in FIG. 7, rats that were administered 30 mg/kg of harmaline demonstrated an average tremor score that was significantly higher than the average tremor score of rats that were administered vehicle and saline only. As also shown in FIG. 7, rats that were administered 10 mg/kg of propranolol demonstrated an average tremor score that was significantly lower than the average tremor score demonstrated by rats that were administered vehicle and harmaline only. Administration to rats of the compound of Formula (I) at the doses of 1, 3 and 10 mg/kg significantly reduced the average tremor score as compared to rats that were administered vehicle and harmaline only.

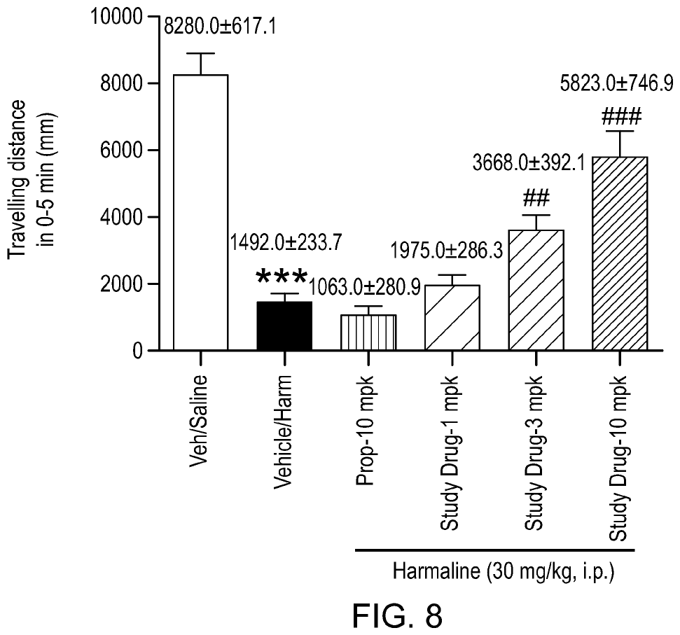

Fifteen minutes after the tremor assessment, the rats were subjected to an open field test. FIG. 8 is a bar graph showing the traveling distance in 0-5 minutes (mm) measured in rats that were administered 30 mg/kg harmaline and also administered 10 mg/kg propranolol or 1 mg/kg, 3 mg/kg or 10 mg/kg study drug. As shown in FIG. 8, rats that were administered 30 mg/kg of harmaline demonstrated a significant reduction in the travelling distance in the open field test as compared to rats that were administered vehicle and saline only. As also shown in FIG. 8, rats that were administered compound of Formula (I) at the dose of 3 and 10 mg/kg demonstrated a significantly attenuated decrease in travelling distance induced by harmaline, as compared to rats that were administered only vehicle and harmaline.

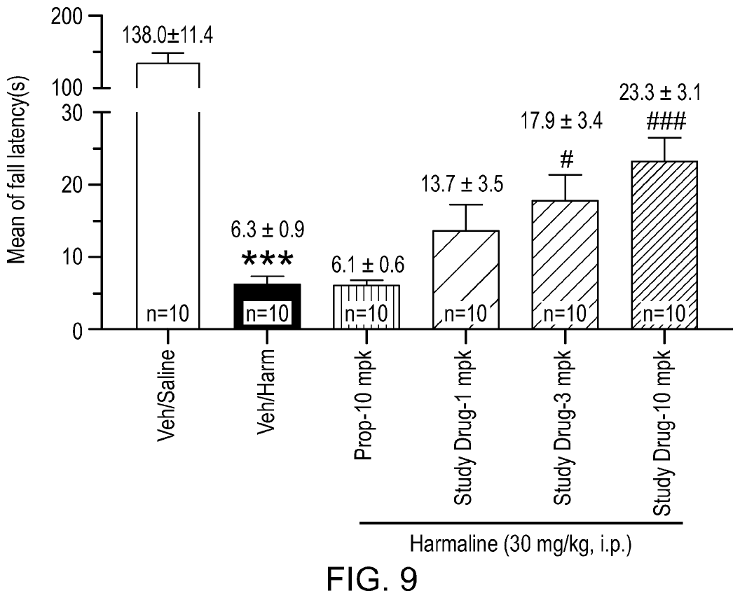

One day before injection of the compound of Formula (I) of vehicle, the rats were trained on a rotarod moving at 8 rpms for five minutes. Fifteen minutes after the open field test, the rats were subjected to a rotarod test. The speed of the rotarod increased from 10 rpms to 60 rpms within the first five minutes of the test. FIG. 9 is a bar graph showing the mean of latency to fall in the rotarod test measured in rats that were administered 30 mg/kg harmaline and also administered 10 mg/kg propranolol or 1 mg/kg, 3 mg/kg or 10 mg/kg study drug. As shown in FIG. 9, rats that were administered 30 mg/kg of harmaline demonstrated a significant motor impairment measured as reduced latency to fall, as compared to rats that were administered only vehicle and saline. As also shown in FIG. 9, rats that were administered the compound of Formula (I), at the dose of 3 and 10 mg/kg demonstrated a significant attenuation of motor impairment (increased latency to fall) as compared to rats that were administered only vehicle and harmaline. By contrast, rats that were administered 10 mg/kg propranolol failed to display any significant improvement in harmaline-induced motor impairment compared to rats that were administered only vehicle and harmaline.

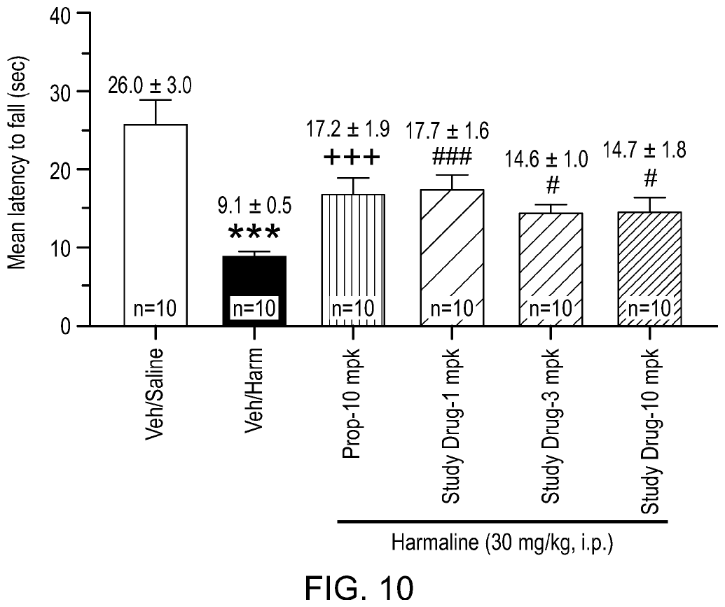

Fifteen minutes after the rotarod test, rats were subjected to a wire grip test using an 80 cm long and 7 mm in diameter wire placed at a height of 50 cm. FIG. 10 is a bar graph showing mean latency to fall (sec) measured in rats that were administered 30 mg/kg harmaline and also administered 10 mg/kg propranolol or 1 mg/kg, 3 mg/kg or 10 mg/kg study drug. As shown in FIG. 10, rats that were administered 30 mg/kg of harmaline demonstrated a significantly reduced latency to fall from the wire as compared to rats that were administered only vehicle and saline. As also shown in FIG. 10, rats that were administered propranolol, as a reference compound, demonstrated a significant reduction in motor impairment (increased latency to fall) as compared to rats that were administered vehicle and saline only. Rats that were administered compound of Formula (I) at the dose of 1, 3 and 10 mg/kg demonstrated a significantly attenuated decrease in fall off latency induced by harmaline, as compared to rats that were administered only vehicle and harmaline.

### CONCLUSIONS

The results of this study demonstrated that administration of 30 mg/kg harmaline successfully induced tremor activity and induced motor impairment in three ADL-like measures. including: a significant reduction in the travelling distance in the open field test and latencies to fall in both rotarod- and wire grip tests in male SD rats. Administration of propranolol had no effect on either the harmaline-induced decrease in spontaneous locomotor activity in the open field (hypolocomotor activity) or latency to fall in the on rotarod test in male SD rats, respectively. By contrast, rats that were administered propranolol demonstrated a significantly inhibited harmaline-induced tremor activity and a significantly attenuated harmaline-induced motor impairment in the wire grip test as compared to rats that were administered only vehicle and harmaline.

Rats that were administered the compound of Formula (I) at the dose of 1, 3 and 10 mg/kg each demonstrated a significant inhibition of tremor activity and significant effects in each of the three ADL-like measures. Formula (I), significantly attenuated the harmaline-induced decrease in travelling distance in the open field and in latencies to fall in the rotarod test and wire grip test as compared to rats that were administered only vehicle and harmaline.

### Example 6. Compound of Formula (I) Co-Dosing with Propranolol in Rat Harmaline-Induced Hypolocomotion

Two harmaline model studies using sLMA as one ADL-like end point were carried out. In each of two sLMA studies, male SD rats were grouped into five groups according to the following table. In the first study, Groups 3, 4, and 5 received 3 mg/kg of propranolol and the compound of Formula (I) (the study drug) at the doses of 1 mg/kg or 3 mg/kg. In the second study, Groups 3, 4, and 5, received 10 mg/kg of propranolol and the study drug at the doses of 1 mg/kg or 3 mg/kg.

TABLE 24

| Group | Administration |
|---|---|
| 1 | Saline + Vehicle |
| 2 | Harmaline + Vehicle |
| 3 | Harmaline + 3 or 10 mg/kg propranolol + Vehicle |
| 4 | Harmaline + 3 or 10 mg/kg propranolol + 1 mg/kg |
| | of compound of Formula (I) |
| 5 | Harmaline + 3 or 10 mg/kg propranolol + 3 mg/kg |
| | of compound of Formula (I) |

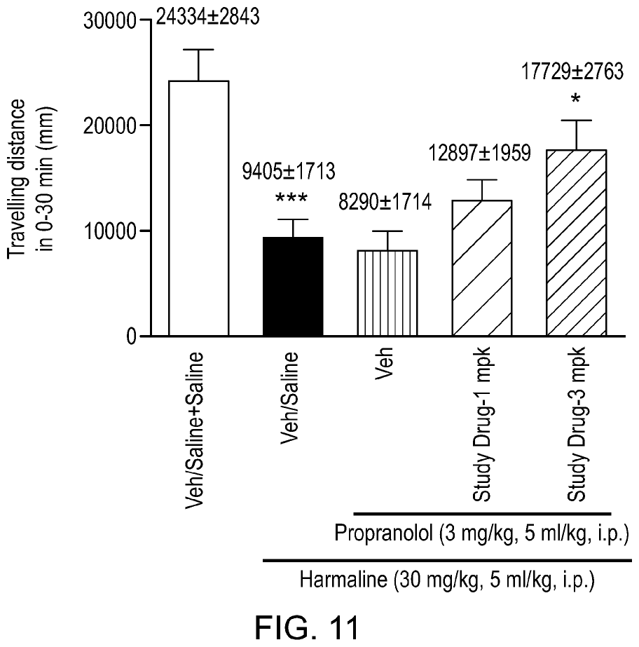

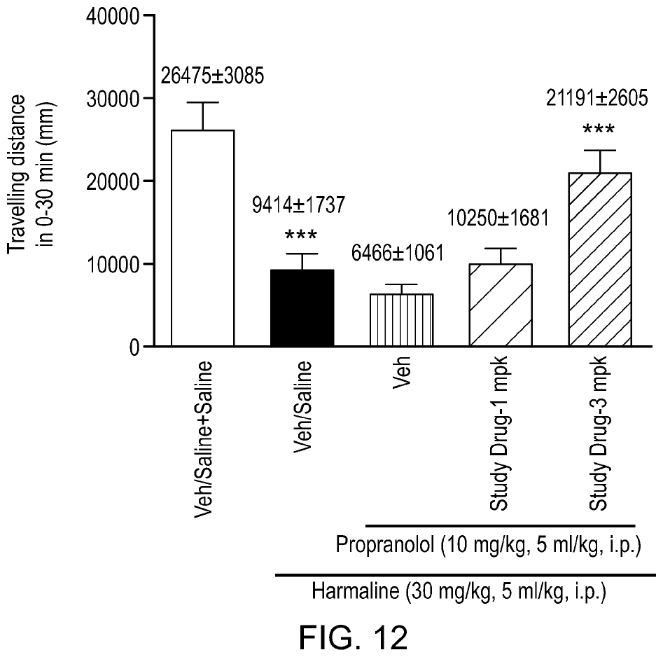

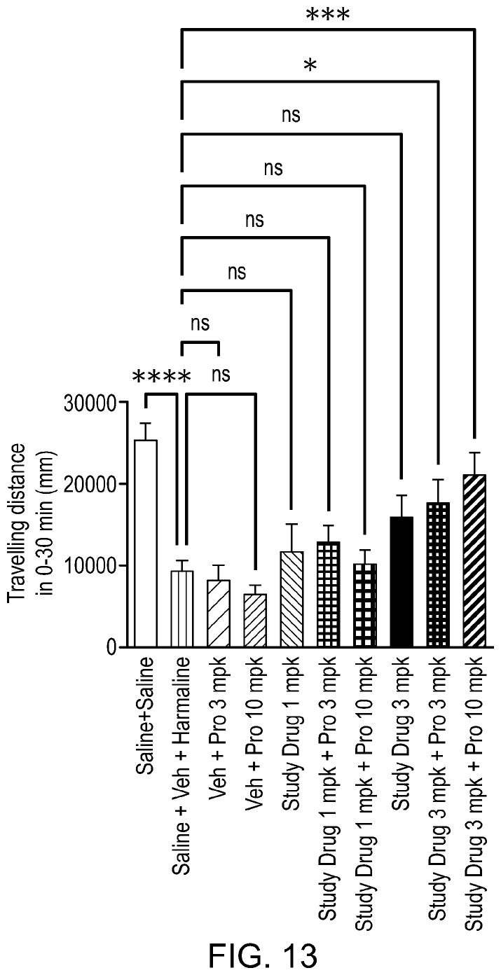

Rats were then subjected to an open field test for 30 minutes. Total distance travelled was measured. The results are presented in FIGS. 11-13. Specifically, FIG. 11 is a bar graph showing traveling distance in 0-30 minutes (mm) of rats treated with harmaline (30 mg/kg) and also treated with 3 mg/kg propranolol and 1 mg/kg and 3 mg/kg of the study drug. As indicated in FIG. 11, there was a significant attenuation of the harmaline-induced hypolomocotion in rats administered both propranolol and study drug at 3 mg/kg, as compared to rats administered vehicle and harmaline only in the first study. FIG. 12 is a bar graph showing traveling distance in 0-30 minutes (mm) of rats treated with harmaline (30 mg/kg) and also treated with 10 mg/kg propranolol and 1 mg/kg and 3 mg/kg of the study drug. FIG. 13 is a bar graph showing the same data as in FIGS. 11 and 12, and also showing the results of statistical comparison between different groups of rats, including data from an identical study from 30 mg/kg harmaline and 1 and 3 mg/kg of the study drug alone (without propranolol). As shown in FIG. 12, in the second study, rats that were administered 10 mg/kg propranolol and 3 mg/kg study drug also demonstrated a significantly increased locomotor activity as compared to the harmaline and vehicle only control rats and were the group of rats to get closest in locomotor activity to rats receiving saline only as shown in FIG. 13. Rats treated with 3 or 10 mg/kg propranolol together with 3 mg/kg study drug showed significant attenuation of harmaline-induce hypolocomotion (relative to rats treated with harmaline and vehicle) but 3 mg/kg study drug alone (without propranolol) was not significantly different from rats treated only with harmaline and vehicle. As such, these results indicate that co-administration of the compound of Formula (I) and propranolol may produce a potentiation of activity in attenuating harmaline-induced hypolocomotion.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The claims or description, thus, include embodiments in which exactly one member of the group is present in, employed in, or otherwise relevant to a given product or process. The claims or description also include embodiments in which more than one, or all of the group members are present in, employed in, or otherwise relevant to a given product or process.

Furthermore, the embodiments encompass all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where embodiments are referred to as comprising particular elements and/or features, certain embodiments may consist of, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a disease or condition relating to aberrant function or activity of a T-type calcium channel in a subject in need thereof, comprising administering to the subject a compound of Formula (I): 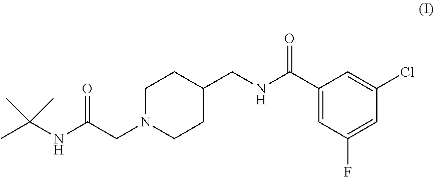 [SMILES: CC(C)(C)NC(=O)CN1CCC(CNC(=O)c2cc(F)cc(Cl)c2)CC1]
or a pharmaceutically acceptable salt thereof in combination with at least one of propranolol, primidone, topiramate, or a pharmaceutically acceptable salt thereof.

2. The method of claim 1, wherein the disease or condition is essential tremor (ET).

3. The method of claim 1 or 2, wherein the compound of Formula (I) is administered as an HCl salt.

4. The method of claim 3, wherein the compound is administered in combination with propranolol.

5. The method of claim 4, wherein the propranolol is administered as an HCl salt.

6. The method of claim 4 or 5, wherein the propranolol is administered as the (S)-enantiomer, the (R)-enantiomer, or a mixture thereof.

7. The method of any one of claims 1-6, wherein the subject was taking a dosing regimen of at least one of propranolol, primidone, topiramate, or a pharmaceutically acceptable salt thereof, prior to administering the compound of Formula (I) or a pharmaceutically acceptable salt thereof.

8. The method of any one of claims 1-7, wherein the dosing regimen of at least one of propranolol, primidone, topiramate, or a pharmaceutically acceptable salt thereof is not altered after administration of the compound of Formula (I) or a pharmaceutically acceptable salt thereof, begins.

9. The method of any one of claims 1-8, wherein the subject is daily administered from about 5 mg to about 120 mg of the compound of Formula (I) or a pharmaceutically acceptable salt thereof.

10. The method of any one of claims 1-8, wherein the subject is daily administered from about 20 mg to about 80 mg of the compound of Formula (I) or a pharmaceutically acceptable salt thereof.

11. The method of any one of claims 1-8, wherein the compound of Formula (I) or a pharmaceutically acceptable salt thereof is titrated upward from a starting dose to a final dose during a course of administration.

12. The method of claim 11, wherein the starting dose of the compound of Formula (I) or a pharmaceutically acceptable salt thereof is between about 10 mg to about 40 mg once daily.

13. The method of claim 11, wherein the starting dose of the compound of Formula (I) or a pharmaceutically acceptable salt thereof is about 20 mg once daily.

14. The method of any one of claims 11-13, wherein the final dose of the compound of Formula (I) or a pharmaceutically acceptable salt thereof is from about 80 mg once daily to about 120 mg once daily.

15. The method of claim 14, wherein the final dose of the compound of Formula (I) or a pharmaceutically acceptable salt thereof is about 120 mg daily.

16. The method of any one of claims 11-15, further comprising administering at least one intermediate dose of the compound of Formula (I) or a pharmaceutically acceptable salt thereof, between the starting dose and the final dose, wherein the amount of the compound of Formula (I) or a pharmaceutically acceptable salt thereof in the intermediate dose is greater than the amount of the compound of Formula (I) or a pharmaceutically acceptable salt thereof in the starting dose but less than the amount of the compound of Formula (I) or a pharmaceutically acceptable salt thereof in the final dose.

17. The method of any one of claims 1-16, wherein the subject is refractory to at least one of propranolol, primidone, topiramate, or a pharmaceutically acceptable salt thereof.

18. The method of any one of claims 1-17, wherein the compound of Formula (I) or a pharmaceutically acceptable salt thereof and the at least one of propranolol, primidone, topiramate, or a pharmaceutically acceptable salt thereof is each administered daily.

19. The method of any one of claims 1-18, wherein the compound of Formula (I) or a pharmaceutically acceptable salt thereof is administered daily and the at least one of propranolol, primidone, topiramate, or a pharmaceutically acceptable salt thereof is administered as needed.

20. The method of any one of claims 1-19, wherein the dose of propranolol, primidone, topiramate is about 5% to 90% lower than the dose of propranolol, primidone, topiramate that the subject received prior to administration the compound of Formula (I) or a pharmaceutically acceptable salt thereof.

21. The method of any one of claims 1-20, wherein the compound of Formula (I) or a pharmaceutically acceptable salt thereof is a deuterium-enriched compound of Formula (I) or pharmaceutically acceptable salt thereof, such as a compounds of formula (IIa) or formula (IIb).

22. A fixed-dose composition for oral administration comprising:
(i) the compound of Formula (I) or a pharmaceutically acceptable salt thereof, and
(ii) at least one of propranolol, primidone, topiramate, or a pharmaceutically acceptable salt thereof.

23. The fixed-dose composition of claim 22, comprising:
(i) the compound of Formula (I) or a pharmaceutically acceptable salt thereof, and
(ii) propranolol or a pharmaceutically acceptable salt thereof.

24. The fixed-dose composition of claim 23, which comprises an HCl salt of the compound of Formula (I) and an HCl salt of propranolol.
